SEQUENCE LISTING (66 sequences)

SEQ ID NO: 1 - RNA, 30 nt, synthetic construct
  source 1..30: mol_type = other RNA
aggggaaccccacagtcgaaccaaaacaaa

SEQ ID NO: 2 - RNA, 30 nt, synthetic construct
  source 1..30: mol_type = other RNA
gacaatatagttctttgagaaggtggaatc

SEQ ID NO: 3 - RNA, 30 nt, synthetic construct
  source 1..30: mol_type = other RNA
gatggagaacaactctaggcagaggtctca

SEQ ID NO: 4 - RNA, 61 nt, Homo sapiens
  source 1..61: mol_type = other RNA
gggtccgccttgagtgcccgggtgagaagcatgatcccgggtaattatggcggacccacag

SEQ ID NO: 5 - RNA, 128 nt, Homo sapiens
  source 1..128: mol_type = other RNA
gtgcacatttcattgacctgctttcttttatgtgagtagtgttatttcttatgtgctatacaaataattgaaggctaattagcagtataactataaatagtaatgctgcctgtgtccttcagacaaaa

SEQ ID NO: 6 - RNA, 28 nt, synthetic construct
  source 1..28: mol_type = other RNA
aaagtgaagaaattcaatgaagcgcggg

SEQ ID NO: 7 - RNA, 24 nt, synthetic construct
  source 1..24: mol_type = other RNA
gaatccgactgtttaattaaaaca

SEQ ID NO: 8 - RNA, 65 nt, synthetic construct
  source 1..65: mol_type = other RNA
ggaattgaaggctggttcgcagtataactataaatagtaatgctgcctgtgtccttcagacaaaa

SEQ ID NO: 9 - RNA, 65 nt, synthetic construct
  source 1..65: mol_type = other RNA
ggaattgaaggcccttctgcagtataactataaatagtaatgctgcaagaacccttcagacaaaa

SEQ ID NO: 10 - RNA, 66 nt, synthetic construct
  source 1..66: mol_type = other RNA
ggaattgaaggctctgccgcagtataactataaatagtaatgctgcgagttgtccttcagacaaaa

SEQ ID NO: 11 - DNA, 5462 bp, synthetic construct
  source 1..5462: mol_type = other DNA
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccacactttatgcttccggctcgtatgttgtgtggaattgtgagcggataacaatttcacacaggaaacagctatgaccatgattacgccaagctcggatcctagagatatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagatcgaattgatcccgcgcagacactgaccttcagcgcctcggctccagcgccatggcttccaaggctcgagctcagtacatcaagagcttcgtggagcgcgtgctgaagaacgagcagtacatggtgcacctgactcctgaggagaagtctgccgttactgccctgtggggcaaggtgaacgtggatgaagttggtggtgaggccctgggcaggtaagtcgacgtgggagattctgcctcggacagagagaaactctgctgtgtctgagagttgatctccctatagtgaccctgccttcttgccccgggacgaaatgagagaaatcgtaacgggagtttacgaggcagacaggttgactctctcttttcccctgcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctgatgggcacctcctgggtttccttcccctggctattctgctcaaccttcctatcagaaaaaaaggggaagcgattctagggagcagtctccatgactgtgtgtggagtgttgacaagagttcggatattttattctctactcagaattgctgctccccctcactctgttctgtgttgtcatttcctctttctttggtaagcttttaatttccagttgcattttactaaattaattaagctggttatttacttcccatcctgatatcagcttcccctcctcctttcctcccagtccttctctctctcctctctctttctctaatcctttcctttccctcagttcatttcttcttctttgatctacgtttgtttgtctttttaaatattgccttgtaacttgctcagaggacaaggaagatatgtccctgtttcttctcatagctctcaagaatagtagcataattggcttttatgccagggtgacaggggaagaatatattttacatataaattctgtttgacataggattcttataataatttgtcagtagtttaaggttgcaaacaaatgtctttgtaaataagcctgcagtatctggtatttttgctctacagttatgttgatggttcttccatattcccacagctcctgggcaatatgatcgtgattgtgctgggccaccacctgggcaaggatttcacccccgctgcacaggctgccttccagaaggtggtggctggagtggccactgccctggctcacaagtaccactaaaccccctttcctgctcttgcctgtgaacaatggttaattgttcccaagagagcatctgtcagttgttggcaaaatgatagacatttgaaaatctgtcttctgacaaataaaaagcatttatgttcactgcaatgatgttttaaattatttgtctgtgtcatagaagggtttatgctaagttttcaagatacaaagaagtgagggttcaggtctgaccttggggaaataaatgaattacacttcaaatgtgtgggacagcaagcagtaagccacagatcctattgccatgccctaaacactcagagaaaaattcaacaaatggtttcatttacacactacattatgattacattttatgtaaattatttgtttttttctactcttccacataaatgtctttttttcctcttacctacccagcacttcacagttctcaagccaataatttttcttttgtaaaactaccattattctctaaacttttccctctgtgtttaccaagcaacattatttatcttttcataaatcctgttgccttagacagcttcagtagcaatagaggtaggattaaggagagaatagaagtgccctgtttgtcataccatgcctgcacagtcaatagtcactatgggatttcaaatggcactttgcctgggacctttacacttcacaccatactctggcttgagttaggagttaagaatgagagaaatataagtctagagagaataagaatatctagtttttaaggctcattactggggtcttatgaaatttccataataccctgtaaatggaagcatttattttttcaataaatctatcttgaatatccagtgtgggttaggattaaatctctccttcatacagttggactgcttttatttatatggagttactagagttaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 12 - DNA, 4974 bp, synthetic construct
  source 1..4974: mol_type = other DNA
ccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattgatctaccatggactacaaagacgatgacgacaagcttatggactacaaggacgacgatgacaagcatatggtgcacctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggatggagaacaactctaggcagaggtctcaaaatttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacccttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtctagaggatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaattccctatagtgagtcgtattaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgaga

SEQ ID NO: 13 - DNA, 32 bp, synthetic construct
  source 1..32: mol_type = other DNA
gtaagtcgacgaattctgcaggctgctggtgg

SEQ ID NO: 14 - DNA, 32 bp, synthetic construct
  source 1..32: mol_type = other DNA
gcctgcagaattcgtcgacttacctgcccagg

SEQ ID NO: 15 - DNA, 21 bp, synthetic construct
  source 1..21: mol_type = other DNA
gttgtcgacgtgggagattct

SEQ ID NO: 16 - DNA, 41 bp, synthetic construct
  source 1..41: mol_type = other DNA
aatctgcaggggaaaagagagagtcaacctgtctgcctcgt

SEQ ID NO: 17 - DNA, 58 bp, synthetic construct
  source 1..58: mol_type = other DNA
tggtggatggagaacaactctaggcagaggtctcaaagtttggggatctgtccactcc

SEQ ID NO: 18 - DNA, 58 bp, synthetic construct
  source 1..58: mol_type = other DNA
ccaaactttgagacctctgcctagagttgttctccatccaccagcagcctaagggtgg

SEQ ID NO: 19 - DNA, 20 bp, synthetic construct
  source 1..20: mol_type = other DNA
tctgccgttactgccctgtg

SEQ ID NO: 20 - DNA, 16 bp, synthetic construct
  source 1..16: mol_type = other DNA
ctttgagacctctgcc

SEQ ID NO: 21 - DNA, 41 bp, synthetic construct
  source 1..41: mol_type = other DNA
aatctgcaggggaaaagagagagtcagtacctgtctgcctc

SEQ ID NO: 22 - DNA, 5464 bp, synthetic construct
  source 1..5464: mol_type = other DNA
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccacactttatgcttccggctcgtatgttgtgtggaattgtgagcggataacaatttcacacaggaaacagctatgaccatgattacgccaagctcggatcctagagatatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagatcgaattgatcccgcgcagacactgaccttcagcgcctcggctccagcgccatggcttccaaggctcgagctcagtacatcaagagcttcgtggagcgcgtgctgaagaacgagcagtacatggtgcacctgactcctgaggagaagtctgccgttactgccctgtggggcaaggtgaacgtggatgaagttggtggtgaggccctgggcaggtaagtcgacgtgggagattctgcctcggacagagagaaactctgctgtgtctgagagttgatctccctatagtgaccctgccttcttgccccgggacgaaatgagagaaatcgtaacgggagtttacgaggcagacaggtactgactctctcttttcccctgcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctgatgggcacctcctgggtttccttcccctggctattctgctcaaccttcctatcagaaaaaaaggggaagcgattctagggagcagtctccatgactgtgtgtggagtgttgacaagagttcggatattttattctctactcagaattgctgctccccctcactctgttctgtgttgtcatttcctctttctttggtaagcttttaatttccagttgcattttactaaattaattaagctggttatttacttcccatcctgatatcagcttcccctcctcctttcctcccagtccttctctctctcctctctctttctctaatcctttcctttccctcagttcatttcttcttctttgatctacgtttgtttgtctttttaaatattgccttgtaacttgctcagaggacaaggaagatatgtccctgtttcttctcatagctctcaagaatagtagcataattggcttttatgccagggtgacaggggaagaatatattttacatataaattctgtttgacataggattcttataataatttgtcagtagtttaaggttgcaaacaaatgtctttgtaaataagcctgcagtatctggtatttttgctctacagttatgttgatggttcttccatattcccacagctcctgggcaatatgatcgtgattgtgctgggccaccacctgggcaaggatttcacccccgctgcacaggctgccttccagaaggtggtggctggagtggccactgccctggctcacaagtaccactaaaccccctttcctgctcttgcctgtgaacaatggttaattgttcccaagagagcatctgtcagttgttggcaaaatgatagacatttgaaaatctgtcttctgacaaataaaaagcatttatgttcactgcaatgatgttttaaattatttgtctgtgtcatagaagggtttatgctaagttttcaagatacaaagaagtgagggttcaggtctgaccttggggaaataaatgaattacacttcaaatgtgtgggacagcaagcagtaagccacagatcctattgccatgccctaaacactcagagaaaaattcaacaaatggtttcatttacacactacattatgattacattttatgtaaattatttgtttttttctactcttccacataaatgtctttttttcctcttacctacccagcacttcacagttctcaagccaataatttttcttttgtaaaactaccattattctctaaacttttccctctgtgtttaccaagcaacattatttatcttttcataaatcctgttgccttagacagcttcagtagcaatagaggtaggattaaggagagaatagaagtgccctgtttgtcataccatgcctgcacagtcaatagtcactatgggatttcaaatggcactttgcctgggacctttacacttcacaccatactctggcttgagttaggagttaagaatgagagaaatataagtctagagagaataagaatatctagtttttaaggctcattactggggtcttatgaaatttccataataccctgtaaatggaagcatttattttttcaataaatctatcttgaatatccagtgtgggttaggattaaatctctccttcatacagttggactgcttttatttatatggagttactagagttaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 23 - DNA, 18 bp, synthetic construct
  source 1..18: mol_type = other DNA
gccataccaccctgaacg

SEQ ID NO: 24 - DNA, 20 bp, synthetic construct
  source 1..20: mol_type = other DNA
agcttccgagatcagacgag

SEQ ID NO: 25 - DNA, 17 bp, synthetic construct
  source 1..17: mol_type = other DNA
ccgagcactttcttgcc

SEQ ID NO: 26 - DNA, 20 bp, synthetic construct
  source 1..20: mol_type = other DNA
aatatggaacgcttcacgaa

SEQ ID NO: 27 - DNA, 51 bp, synthetic construct
  source 1..51: mol_type = other DNA
attaagcttgtgtgggagattcttcttcggacagagagaaactctgctgtg

SEQ ID NO: 28 - DNA, 60 bp, synthetic construct
  source 1..60: mol_type = other DNA
ctgctgtgtctgaaagaagatctccctatagtgaccctgccttaccttctccgggacgaa

SEQ ID NO: 29 - DNA, 58 bp, synthetic construct
  source 1..58: mol_type = other DNA
atggatccacctgtctgcctcgtattcttccgttacgatttctctcatttcgtcccgg

SEQ ID NO: 30 - DNA, 51 bp, synthetic construct
  source 1..51: mol_type = other DNA
attaagcttgtgtgggagattctgcctcggacagagagaaactctgctgtg

SEQ ID NO: 31 - DNA, 60 bp, synthetic construct
  source 1..60: mol_type = other DNA
ttcgtcccggggcagagaaggcagggtcactatagggagatcaactctcagacacagcag

SEQ ID NO: 32 - DNA, 58 bp, synthetic construct
  source 1..58: mol_type = other DNA
atggatccacctgtctgcctcgtaaactcccgttacgatttctctcatttcgtcccgg

SEQ ID NO: 33 - DNA, 58 bp, synthetic construct
  source 1..58: mol_type = other DNA
tggtggacaatatagttctttgagaaggtggaatcacatttggggatctgtccactcc

SEQ ID NO: 34 - DNA, 58 bp, synthetic construct
  source 1..58: mol_type = other DNA
ccaaatgtgattccaccttctcaaagaactatattgtccaccagcagcctaagggtgg

SEQ ID NO: 35 - DNA, 16 bp, synthetic construct
  source 1..16: mol_type = other DNA
ctggagccttcagagg

SEQ ID NO: 36 - DNA, 19 bp, synthetic construct
  source 1..19: mol_type = other DNA
gctcttgctaaagaaattc

SEQ ID NO: 37 - RNA, 65 nt, synthetic construct; identical to SEQ ID NO: 8
  source 1..65: mol_type = other RNA
  modified_base 1..2: mod_base = OTHER; note = 2'-OMe
  modified_base 8..20: mod_base = OTHER; note = 2'-OMe
  modified_base 23..31: mod_base = OTHER; note = 2'-OMe
  modified_base 38..44: mod_base = OTHER; note = 2'-OMe
  modified_base 47: mod_base = OTHER; note = 2'-OMe
  modified_base 51..56: mod_base = OTHER; note = 2'-OMe
  modified_base 59: mod_base = OTHER; note = 2'-OMe
  modified_base 61: mod_base = OTHER; note = 2'-OMe
  modified_base 64..65: mod_base = OTHER; note = 2'-OMe
  modified_base 1..7: mod_base = OTHER; note = phosphorothioate modification
  modified_base 21..22: mod_base = OTHER; note = phosphorothioate modification
  modified_base 31..33: mod_base = OTHER; note = phosphorothioate modification
  modified_base 35..36: mod_base = OTHER; note = phosphorothioate modification
  modified_base 44..45: mod_base = OTHER; note = phosphorothioate modification
  modified_base 47..49: mod_base = OTHER; note = phosphorothioate modification
  modified_base 56: mod_base = OTHER; note = phosphorothioate modification
  modified_base 58: mod_base = OTHER; note = phosphorothioate modification
  modified_base 61..64: mod_base = OTHER; note = phosphorothioate modification

SEQ ID NO: 38 - RNA, 65 nt, synthetic construct; identical to SEQ ID NO: 8
  source 1..65: mol_type = other RNA
  modified_base 1..2: mod_base = OTHER; note = 2'-OMe
  modified_base 8..20: mod_base = OTHER; note = 2'-OMe
  modified_base 23..31: mod_base = OTHER; note = 2'-OMe
  modified_base 38..44: mod_base = OTHER; note = 2'-OMe
  modified_base 47: mod_base = OTHER; note = 2'-OMe
  modified_base 51..56: mod_base = OTHER; note = 2'-OMe
  modified_base 59: mod_base = OTHER; note = 2'-OMe
  modified_base 61: mod_base = OTHER; note = 2'-OMe
  modified_base 64..65: mod_base = OTHER; note = 2'-OMe
  modified_base 1..4: mod_base = OTHER; note = phosophorothioate modification
  modified_base 61..64: mod_base = OTHER; note = phosophorothioate modification

SEQ ID NO: 39 - RNA, 65 nt, synthetic construct; identical to SEQ ID NO: 8
  source 1..65: mol_type = other RNA
  modified_base 1..2: mod_base = OTHER; note = 2'-OMe
  modified_base 8..20: mod_base = OTHER; note = 2'-OMe
  modified_base 23..26: mod_base = OTHER; note = 2'-OMe
  modified_base 38..44: mod_base = OTHER; note = 2'-OMe
  modified_base 51..56: mod_base = OTHER; note = 2'-OMe
  modified_base 64..65: mod_base = OTHER; note = 2'-OMe
  modified_base 1..3: mod_base = OTHER; note = phosphorothioate modification
  modified_base 63..65: mod_base = OTHER; note = phosphorothioate modification

SEQ ID NO: 40 - RNA, 65 nt, synthetic construct; identical to SEQ ID NO: 8
  source 1..65: mol_type = other RNA
  modified_base 1..2: mod_base = OTHER; note = 2'-OMe
  modified_base 8..11: mod_base = OTHER; note = 2'-OMe
  modified_base 23..26: mod_base = OTHER; note = 2'-OMe
  modified_base 38..44: mod_base = OTHER; note = 2'-OMe
  modified_base 53..56: mod_base = OTHER; note = 2'-OMe
  modified_base 64..65: mod_base = OTHER; note = 2'-OMe
  modified_base 1..3: mod_base = OTHER; note = phosphorothioate modification
  modified_base 63..65: mod_base = OTHER; note = phosphorothioate modification

SEQ ID NO: 41 - skipped

SEQ ID NO: 42 - RNA, 65 nt, synthetic construct; identical to SEQ ID NO: 9
  source 1..65: mol_type = other RNA
  modified_base 1..2: mod_base = OTHER; note = 2'-OMe
  modified_base 8..20: mod_base = OTHER; note = 2'-OMe
  modified_base 23..31: mod_base = OTHER; note = 2'-OMe
  modified_base 38..44: mod_base = OTHER; note = 2'-OMe
  modified_base 47: mod_base = OTHER; note = 2'-OMe
  modified_base 51..56: mod_base = OTHER; note = 2'-OMe
  modified_base 59: mod_base = OTHER; note = 2'-OMe
  modified_base 61: mod_base = OTHER; note = 2'-OMe
  modified_base 64..65: mod_base = OTHER; note = 2'-OMe
  modified_base 1..4: mod_base = OTHER; note = phosphorothioate modification
  modified_base 61..64: mod_base = OTHER; note = phosphorothioate modification

SEQ ID NO: 43 - RNA, 65 nt, synthetic construct; identical to SEQ ID NO: 9
  source 1..65: mol_type = other RNA
  modified_base 1..2: mod_base = OTHER; note = 2'-OMe
  modified_base 8..20: mod_base = OTHER; note = 2'-OMe
  modified_base 23..26: mod_base = OTHER; note = 2'-OMe
  modified_base 38..44: mod_base = OTHER; note = 2'-OMe
  modified_base 64..65: mod_base = OTHER; note = 2'-OMe
  modified_base 51..56: mod_base = OTHER; note = 2'-OMe
  modified_base 1..3: mod_base = OTHER; note = phosphorothioate modification
  modified_base 62..64: mod_base = OTHER; note = phosphorothioate modification

SEQ ID NO: 44 - RNA, 65 nt, synthetic construct; identical to SEQ ID NO: 9
  source 1..65: mol_type = other RNA
  modified_base 1..2: mod_base = OTHER; note = 2'-OMe
  modified_base 8..11: mod_base = OTHER; note = 2'-OMe
  modified_base 19..20: mod_base = OTHER; note = 2'-OMe
  modified_base 23..26: mod_base = OTHER; note = 2'-OMe
  modified_base 38..44: mod_base = OTHER; note = 2'-OMe
  modified_base 53..56: mod_base = OTHER; note = 2'-OMe
  modified_base 64..65: mod_base = OTHER; note = 2'-OMe
  modified_base 1..3: mod_base = OTHER; note = phosphorothioate modification
  modified_base 62..64: mod_base = OTHER; note = phosphorothioate modification

SEQ ID NO: 45 - RNA, 66 nt, synthetic construct; identical to SEQ ID NO: 10
  source 1..66: mol_type = other RNA
  modified_base 1..2: mod_base = OTHER; note = 2'-OMe
  modified_base 8..20: mod_base = OTHER; note = 2'-OMe
  modified_base 23..31: mod_base = OTHER; note = 2'-OMe
  modified_base 38..44: mod_base = OTHER; note = 2'-OMe
  modified_base 47: mod_base = OTHER; note = 2'-OMe
  modified_base 52..57: mod_base = OTHER; note = 2'-OMe
  modified_base 60: mod_base = OTHER; note = 2'-OMe
  modified_base 62: mod_base = OTHER; note = 2'-OMe
  modified_base 65..66: mod_base = OTHER; note = 2'-OMe
  modified_base 1..7: mod_base = OTHER; note = phosphorothioate modification
  modified_base 21..22: mod_base = OTHER; note = phosphorothioate modification
  modified_base 31..33: mod_base = OTHER; note = phosphorothioate modification
  modified_base 35..36: mod_base = OTHER; note = phosphorothioate modification
  modified_base 44..45: mod_base = OTHER; note = phosphorothioate modification
  modified_base 47..50: mod_base = OTHER; note = phosphorothioate modification
  modified_base 57: mod_base = OTHER; note = phosphorothioate modification
  modified_base 59: mod_base = OTHER; note = phosphorothioate modification
  modified_base 62..65: mod_base = OTHER; note = phosphorothioate modification

SEQ ID NO: 46 - RNA, 66 nt, synthetic construct; identical to SEQ ID NO: 10
  source 1..66: mol_type = other RNA
  modified_base 1..2: mod_base = OTHER; note = 2'-OMe
  modified_base 8..20: mod_base = OTHER; note = 2'-OMe
  modified_base 23..31: mod_base = OTHER; note = 2'-OMe
  modified_base 38..44: mod_base = OTHER; note = 2'-OMe
  modified_base 47: mod_base = OTHER; note = 2'-OMe
  modified_base 52..57: mod_base = OTHER; note = 2'-OMe
  modified_base 60: mod_base = OTHER; note = 2'-OMe
  modified_base 62: mod_base = OTHER; note = 2'-OMe
  modified_base 65..66: mod_base = OTHER; note = 2'-OMe
  modified_base 1..4: mod_base = OTHER; note = phosphorothioate modification
  modified_base 62..65: mod_base = OTHER; note = phosphorothioate modification

SEQ ID NO: 47 - RNA, 66 nt, synthetic construct; identical to SEQ ID NO: 10
  source 1..66: mol_type = other RNA
  modified_base 1..2: mod_base = OTHER; note = 2'-OMe
  modified_base 8..20: mod_base = OTHER; note = 2'-OMe
  modified_base 23..26: mod_base = OTHER; note = 2'-OMe
  modified_base 38..44: mod_base = OTHER; note = 2'-OMe
  modified_base 52..57: mod_base = OTHER; note = 2'-OMe
  modified_base 65..66: mod_base = OTHER; note = 2'-OMe
  modified_base 1..3: mod_base = OTHER; note = phosphorothioate modification
  modified_base 63..65: mod_base = OTHER; note = phosphorothioate modification

SEQ ID NO: 48 - RNA, 66 nt, synthetic construct; identical to SEQ ID NO: 10
  source 1..66: mol_type = other RNA
  modified_base 1..2: mod_base = OTHER; note = 2'-OMe
  modified_base 8..11: mod_base = OTHER; note = 2'-OMe
  modified_base 19..20: mod_base = OTHER; note = 2'-OMe
  modified_base 23..26: mod_base = OTHER; note = 2'-OMe
  modified_base 38..44: mod_base = OTHER; note = 2'-OMe
  modified_base 54..57: mod_base = OTHER; note = 2'-OMe
  modified_base 65..66: mod_base = OTHER; note = 2'-OMe
  modified_base 1..3: mod_base = OTHER; note = phosphorothioate modification
  modified_base 63..65: mod_base = OTHER; note = phosphorothioate modification

SEQ ID NO: 49 - RNA, 25 nt, synthetic construct
  source 1..25: mol_type = other RNA
ggaattgaaggcccttctgcagtat

SEQ ID NO: 50 - RNA, 40 nt, synthetic construct
  source 1..40: mol_type = other RNA
aactataaatagtaatgctgcaagaacccttcagacaaaa

SEQ ID NO: 51 - RNA, 38 nt, synthetic construct
  source 1..38: mol_type = other RNA
ggaattgaaggcccttctgcagtataactataaatagt

SEQ ID NO: 52 - RNA, 27 nt, synthetic construct
  source 1..27: mol_type = other RNA
aatgctgcaagaacccttcagacaaaa

SEQ ID NO: 53 - RNA, 32 nt, synthetic construct
  source 1..32: mol_type = other RNA
ggaattgaaggcccttctgcagtataactata

SEQ ID NO: 54 - RNA, 33 nt, synthetic construct
  source 1..33: mol_type = other RNA
aatagtaatgctgcaagaacccttcagacaaaa

SEQ ID NO: 55 - RNA, 65 nt, synthetic construct; identical to SEQ ID NO: 9
  source 1..65: mol_type = other RNA
  modified_base 1..2: mod_base = OTHER; note = 2'-OMe
  modified_base 8..20: mod_base = OTHER; note = 2'-OMe
  modified_base 23..31: mod_base = OTHER; note = 2'-OMe
  modified_base 38..44: mod_base = OTHER; note = 2'-OMe
  modified_base 47: mod_base = OTHER; note = 2'-OMe
  modified_base 51..56: mod_base = OTHER; note = 2'-OMe
  modified_base 58..59: mod_base = OTHER; note = 2'-OMe
  modified_base 61: mod_base = OTHER; note = 2'-OMe
  modified_base 64..65: mod_base = OTHER; note = 2'-OMe
  modified_base 1..7: mod_base = OTHER; note = phosphorothioate modifications
  modified_base 21..22: mod_base = OTHER; note = phosphorothioate modifications
  modified_base 31..33: mod_base = OTHER; note = phosphorothioate modifications
  modified_base 44..45: mod_base = OTHER; note = phosphorothioate modifications
  modified_base 47..49: mod_base = OTHER; note = phosphorothioate modifications
  modified_base 56: mod_base = OTHER; note = phosphorothioate modifications
  modified_base 59: mod_base = OTHER; note = phosphorothioate modifications
  modified_base 61..64: mod_base = OTHER; note = phosphorothioate modifications

SEQ ID NO: 56 - RNA, 65 nt, synthetic construct; identical to SEQ ID NO: 9
  source 1..65: mol_type = other RNA
  modified_base 1..2: mod_base = OTHER; note = 2'-OMe
  modified_base 8..20: mod_base = OTHER; note = 2'-OMe
  modified_base 23..31: mod_base = OTHER; note = 2'-OMe
  modified_base 38..44: mod_base = OTHER; note = 2'-OMe
  modified_base 47: mod_base = OTHER; note = 2'-OMe
  modified_base 52..56: mod_base = OTHER; note = 2'-OMe
  modified_base 59: mod_base = OTHER; note = 2'-OMe
  modified_base 61: mod_base = OTHER; note = 2'-OMe
  modified_base 64..65: mod_base = OTHER; note = 2'-OMe
  modified_base 1..7: mod_base = OTHER; note = phosphorothioate modification
  modified_base 21..22: mod_base = OTHER; note = phosphorothioate modification
  modified_base 31..33: mod_base = OTHER; note = phosphorothioate modification
  modified_base 35..36: mod_base = OTHER; note = phosphorothioate modification
  modified_base 44..45: mod_base = OTHER; note = phosphorothioate modification
  modified_base 47..50: mod_base = OTHER; note = phosphorothioate modification
  modified_base 56: mod_base = OTHER; note = phosphorothioate modification
  modified_base 59: mod_base = OTHER; note = phosphorothioate modification
  modified_base 61..64: mod_base = OTHER; note = phosphorothioate modification

SEQ ID NO: 57 - RNA, 32 nt, synthetic construct; identical to SEQ ID NO: 53
  source 1..32: mol_type = other RNA
  modified_base 1..2: mod_base = OTHER; note = 2'-OMe
  modified_base 8..20: mod_base = OTHER; note = 2'-OMe
  modified_base 23..30: mod_base = OTHER; note = 2'-OMe
  modified_base 1..7: mod_base = OTHER; note = phosphorothioate modification
  modified_base 21..22: mod_base = OTHER; note = phosphorothioate modification
  modified_base 31: mod_base = OTHER; note = phosphorothioate modification

SEQ ID NO: 58 - RNA, 33 nt, synthetic construct; identical to SEQ ID NO: 54
  source 1..33: mol_type = other RNA
  modified_base 1: mod_base = OTHER; note = phosphorothioate modification
  modified_base 3..4: mod_base = OTHER; note = phosphorothioate modification
  modified_base 12..13: mod_base = OTHER; note = phosphorothioate modification
  modified_base 15..17: mod_base = OTHER; note = phosphorothioate modification
  modified_base 24: mod_base = OTHER; note = phosphorothioate modification
  modified_base 27: mod_base = OTHER; note = phosphorothioate modification
  modified_base 29..32: mod_base = OTHER; note = phosphorothioate modification
  modified_base 6..12: mod_base = OTHER; note = 2'-OMe
  modified_base 15: mod_base = OTHER; note = 2'-OMe
  modified_base 19..24: mod_base = OTHER; note = 2'-OMe
  modified_base 26..27: mod_base = OTHER; note = 2'-OMe
  modified_base 29: mod_base = OTHER; note = 2'-OMe
  modified_base 32..33: mod_base = OTHER; note = 2'-OMe

SEQ ID NO: 59 - RNA, 32 nt, synthetic construct; identical to SEQ ID NO: 53
  source 1..32: mol_type = other RNA
  modified_base 1..2: mod_base = OTHER; note = 2'-OMe
  modified_base 8..20: mod_base = OTHER; note = 2'-OMe
  modified_base 23..31: mod_base = OTHER; note = 2'-OMe
  modified_base 1..7: mod_base = OTHER; note = phosphorothioate modification
  modified_base 21..22: mod_base = OTHER; note = phosphorothioate modification
  modified_base 31: mod_base = OTHER; note = phosphorothioate modification

SEQ ID NO: 60 - RNA, 33 nt, synthetic construct; identical to SEQ ID NO: 54
  source 1..33: mol_type = other RNA
  modified_base 6..12: mod_base = OTHER; note = 2'-OMe
  modified_base 15: mod_base = OTHER; note = 2'-OMe
  modified_base 20..24: mod_base = OTHER; note = 2'-OMe
  modified_base 27: mod_base = OTHER; note = 2'-OMe
  modified_base 29: mod_base = OTHER; note = 2'-OMe
  modified_base 32..33: mod_base = OTHER; note = 2'-OMe
  modified_base 1: mod_base = OTHER; note = phosphorothioate modification
  modified_base 3..4: mod_base = OTHER; note = phosphorothioate modification
  modified_base 12..13: mod_base = OTHER; note = phosphorothioate modification
  modified_base 15..18: mod_base = OTHER; note = phosphorothioate modification
  modified_base 24: mod_base = OTHER; note = phosphorothioate modification
  modified_base 27: mod_base = OTHER; note = phosphorothioate modification
  modified_base 29..32: mod_base = OTHER; note = phosphorothioate modification

SEQ ID NO: 61 - RNA, 32 nt, synthetic construct
  source 1..32: mol_type = other RNA
  modified_base 1..2: mod_base = OTHER; note = 2'-OMe
  modified_base 8..20: mod_base = OTHER; note = 2'OMe
  modified_base 23..31: mod_base = OTHER; note = 2'-OMe
  modified_base 1..7: mod_base = OTHER; note = phosphorothioate modifications
  modified_base 21..22: mod_base = OTHER; note = phosphorothioate modifications
  modified_base 31: mod_base = OTHER; note = phosphorothioate modifications
ggaattgaaggcccttctgcagtataactatg

SEQ ID NO: 62 - RNA, 33 nt, synthetic construct
  source 1..33: mol_type = other RNA
  modified_base 6..12: mod_base = OTHER; note = 2'-OMe
  modified_base 15: mod_base = OTHER; note = 2'-OMe
  modified_base 20..24: mod_base = OTHER; note = 2'-OMe
  modified_base 27: mod_base = OTHER; note = 2'-OMe
  modified_base 29: mod_base = OTHER; note = 2'-OMe
  modified_base 32..33: mod_base = OTHER; note = 2'-OMe
  modified_base 1: mod_base = OTHER; note = phosphorothioate modifications
  modified_base 3..4: mod_base = OTHER; note = phosphorothioate modifications
  modified_base 12..13: mod_base = OTHER; note = phosphorothioate modifications
  modified_base 15..18: mod_base = OTHER; note = phosphorothioate modifications
  modified_base 24: mod_base = OTHER; note = phosphorothioate modifications
  modified_base 27: mod_base = OTHER; note = phosphorothioate modifications
  modified_base 29..32: mod_base = OTHER; note = phosphorothioate modifications
catagtaatgctgcaagaacccttcagacaaaa

SEQ ID NO: 63 - RNA, 32 nt, synthetic construct; identical to SEQ ID NO: 61
  source 1..32: mol_type = other RNA
  modified_base 1..2: mod_base = OTHER; note = 2'-OMe
  modified_base 8..20: mod_base = OTHER; note = 2'-OMe
  modified_base 23..32: mod_base = OTHER; note = 2'-OMe
  modified_base 1..7: mod_base = OTHER; note = phosphorothioate modification
  modified_base 21..22: mod_base = OTHER; note = phosphorothioate modification
  modified_base 31: mod_base = OTHER; note = phosphorothioate modification

SEQ ID NO: 64 - RNA, 33 nt, synthetic construct; identical to SEQ ID NO: 62
  source 1..33: mol_type = other RNA
  modified_base 1: mod_base = OTHER; note = 2'-OMe
  modified_base 6..12: mod_base = OTHER; note = 2'-OMe
  modified_base 15: mod_base = OTHER; note = 2'-OMe
  modified_base 20..24: mod_base = OTHER; note = 2'-OMe
  modified_base 27: mod_base = OTHER; note = 2'-OMe
  modified_base 29: mod_base = OTHER; note = 2'-OMe
  modified_base 32..33: mod_base = OTHER; note = 2'-OMe
  modified_base 1: mod_base = OTHER; note = phosphorothioate modification
  modified_base 3..4: mod_base = OTHER; note = phosphorothioate modification
  modified_base 12..13: mod_base = OTHER; note = phosphorothioate modification
  modified_base 15..18: mod_base = OTHER; note = phosphorothioate modification
  modified_base 24: mod_base = OTHER; note = phosphorothioate modification
  modified_base 27: mod_base = OTHER; note = phosphorothioate modification
  modified_base 29..32: mod_base = OTHER; note = phosphorothioate modification

SEQ ID NO: 65 - RNA, 32 nt, synthetic construct
  source 1..32: mol_type = other RNA
  modified_base 1..2: mod_base = OTHER; note = 2'-OMe
  modified_base 8..20: mod_base = OTHER; note = 2'-OMe
  modified_base 23..31: mod_base = OTHER; note = 2'-OMe
  modified_base 1..7: mod_base = OTHER; note = phosphorothioate modification
  modified_base 21..22: mod_base = OTHER; note = phosphorothioate modification
  modified_base 31: mod_base = OTHER; note = phosphorothioate modification
ggaattgaaggcccttctgcagtataactgtg

SEQ ID NO: 66 - RNA, 33 nt, synthetic construct
  source 1..33: mol_type = other RNA
  modified_base 6..12: mod_base = OTHER; note = 2'-OMe
  modified_base 15: mod_base = OTHER; note = 2'-OMe
  modified_base 20..24: mod_base = OTHER; note = 2'-OMe
  modified_base 27: mod_base = OTHER; note = 2'-OMe
  modified_base 29: mod_base = OTHER; note = 2'-OMe
  modified_base 32..33: mod_base = OTHER; note = 2'-OMe
  modified_base 1: mod_base = OTHER; note = phosphorothioate modification
  modified_base 3..4: mod_base = OTHER; note = phosphorothioate modification
  modified_base 12..13: mod_base = OTHER; note = phosphorothioate modification
  modified_base 15..18: mod_base = OTHER; note = phosphorothioate modification
  modified_base 24: mod_base = OTHER; note = phosphorothioate modification
  modified_base 27: mod_base = OTHER; note = phosphorothioate modification
  modified_base 29..32: mod_base = OTHER; note = phosphorothioate modification
cacagtaatgctgcaagaacccttcagacaaaa

(19) United States
(12) Patent Application Publication
(10) Pub. No.: US 2026/0297577 A1
(43) Pub. Date: Oct. 1, 2026

(54) Nucleic Acid Molecules for Pseudouridylation

(71) Applicant: University of Rochester, Rochester, NY (US)

(72) Inventors: Peter Christian De Visser, CK Leiden (NL); Pedro Duarte Morais Fernandes Arantes da Silva, CK Leiden (NL); Yi-Tao Yu, Rochester, NY (US); Jonathan Chen, Rochester, NY (US)

(21) Appl. No.: 19/477,161

(22) PCT Filed: Apr. 19, 2024
(86) PCT No.: PCT/US2024/025581

§ 371 Date: Oct. 21, 2025

Related U.S. Application Data
(60) Provisional application No. 63/497,497, filed on Apr. 21, 2023.

(51) Int. Cl. C12N 15/113

(52) U.S. Cl. CPC C12N 15/113; C12N 2310/315; C12N 2310/321; C12N 2310/322; C12N 2310/531

(57) ABSTRACT

The invention relates to combinations of oligonucleotides that hybridize to form a partially-duplexed nucleic acid structure comprising a guide region capable of forming a complex with a target RNA for pseudouridylation of the target uridine in a target RNA in a cell, wherein complex is capable of engaging a pseudouridylation enzyme, wherein the guide region assists in positioning the target uridine in the complex for it to be converted to a pseudouridine by the pseudouridylation enzyme.

### CROSS-REFERENCE TO RELATED APPLICATIONS

This application claims priority to and the benefit of U.S. Provisional Application No. 63/497,497 filed Apr. 21, 2023, the disclosure of which is incorporated herein by reference in its entirety.

### REFERENCE TO A “SEQUENCE LISTING” SUBMITTED AS AN XML FILE

The present application hereby incorporates by reference the entire contents of the XML file named “204606-0163-OOWO_SequenceListing” which was created on Apr. 19, 2024 and is 212,579 bytes in size.

### FIELD OF THE INVENTION

The invention relates to the field of medicine. More in particular, the invention relates to the field of pseudouridylation, whereby an RNA molecule in a cell is targeted by a nucleic acid molecule, such as an oligonucleotide, to recruit a pseudouridine synthase to convert a specific uridine present in the RNA sequence into pseudouridine. More specifically, the invention relates to oligonucleotides and intron-embedded snoRNAs that promote pseudouridylation of a uridine in a target RNA and methods of use thereof.

### BACKGROUND OF THE INVENTION

Pseudouridine (ψ) is the most abundant post-transcriptionally modified nucleotide in stable RNAs, including tRNA, rRNA, snRNA and mRNA, constituting approximately 5% of total ribonucleotides. The conversion of uridine to ψ (pseudouridylation) requires two distinct chemical reactions: the breaking of the C1′-N1 glycosydic bond and the making of a new carbon (C1′-C5) bond that relinks the base to the sugar. Pseudouridylation is a true isomerization reaction, which creates an extra hydrogen bond donor and thereby influences a wide variety of functional aspects depending on the type of RNA that carries the ψ and the position within the RNA sequence, such as protein synthesis, increased stop-codon read-through and frame shifting (Yu and Meier, 2014, RNA Biology 11:1483-1494). FIG. 1 shows the structures of uridine and ψ. Many of the mRNA ψs reside in coding regions, and the majority of them respond to environmental stress, indicating functional significance (Carlile et al. 2014, Nature 515:143-146).

In eukaryotes and archaea, pseudouridylation is introduced among other proteins by box H/ACA ribonucleoproteins (RNPs), each of which contains a unique small RNA (box H/ACA RNA, one of the two major classes of small nucleolar RNAs, or ‘snoRNAs’) and four core proteins (NAP75/dyskerin/Cbf5, Nhp2, Nop10 and Gar1). NAP75/dyskerin/Cbf5 catalyses the chemical reactions, converting the target uridine to ψ. The RNA component serves as a guide that specifies, through base-pairing interaction with its substrate RNA, the target uridine for pseudouridylation (Ge and Yu, 2013, Trends Biochem Sci 38(4):210-218). Based on this guide-substrate base-pairing scheme, Karijolich and Yu (2011, Nature 474:395-398) designed an artificial box H/ACA RNA to introduce ψ into mRNA at a Premature Termination Codon (PTC) in S. cerevisiae. They demonstrated that ψ was indeed incorporated into TRM4 mRNA at the PTC. Remarkably, pseudouridylated PTC promoted nonsense suppression by altering ribosome decoding (Fernandez et al. 2013, Nature 500:107-110; Wu et al. 2015, Methods in Enzymology 560:187-217; U.S. Pat. No. 8,603,457). Using a similar strategy, others showed that artificial H/ACA RNAs could site-specifically pseudouridylate pre-mRNA after microinjection into Xenopus oocytes (Chen et al. 2010, Mol Cell Biol 30:4108-4119). In both examples, the artificial H/ACA RNAs were modified to alter the loops that serve as the guide sequence, but otherwise these snoRNAs were unaltered and still full length.

Mammalian H/ACA snoRNAs are generally embedded (positioned) within pre-mRNA intronic regions of protein-coding genes. During transcription elongation, several proteins with a functional role in pseudouridylation, such as Nop10, dyskerin or Nhp2 bind to the nascent H/ACA snoRNA sequences, particularly to their structural core motifs. Following splicing, the guide RNAs are processed through debranching and exonucleolytic processing, resulting in a RNA-protein complex called ‘small nuclear ribonucleoproteins’ (snRNPs, or snRNP complex). Once in the mature form, these particles are intra-nuclearly trafficked from the transcription sites to the site where they are functionally active for pseudouridylation, mostly in the nucleolus but also in the Cajal bodies. During spliceosome-assembly these snRNPs are sequentially recruited on to a pre-mRNA substrate, onto which short RNA-RNA hybrids will form allowing specification of the pseudouridylation sites. Box H/ACA snoRNAs have no preference for localization relative to the 5′ or 3′ ends of the intron and can be present in small or very large introns, as opposed to box C/D snoRNAs, which are usually localized 60-90 nucleotides upstream the 3′-splice site and are encoded in relatively small introns. Leverette et al. (1992, Cell 71(7):1215-1221) proposed that some snoRNAs could be present in intronic regions of pre-mRNAs. Kiss and Filipowicz (1995, Genes Dev 9(11):1411-1424) suggested that a given snoRNA sequence could be excised and fully processed from an intronic region of any given actively spliced mRNA. To show the feasibility of this approach, they artificially imbedded several snoRNAs (U17a, U17b and U19) into the second intron of the human β-globin gene and expressed the resulting vector in fibroblast-like cells. After transfection, they found that the artificial, intronically delivered snoRNAs were properly processed from the human β-globin intron and the globin pre-mRNA was correctly spliced. Notably, this did not happen when the guide RNA sequence was artificially embedded in an exon. Darzacq et al. (2002, EMBO J 21(11);2746-2756) corroborated that other guide RNAs could be inserted into the second intron of the human β-globin gene using an expression vector under the control of the cytomegalovirus (CMV) promoter and be delivered to mammalian cells via transfection.

In spite of the above, and the fact that snoRNAs could be delivered to cells and be processed from intronic regions, no one—to the knowledge of the inventors of the present invention—has ever shown targeted pseudouridylation using snoRNAs in a mammalian system. The artificial H/ACA snoRNAs described in the prior art are typically in the range of about 140 nt and they consist of RNA, which makes that they tend to be quite unstable in vivo. Manufacturing and delivery of such molecules in a therapeutic setting therefore remains a challenge.

### SUMMARY OF THE INVENTION

In some embodiments, the invention relates to a composition comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme.

In one embodiment, the pseudouridylation enzyme is part of a ribonucleoprotein (RNP) complex capable of acting on an H/ACA-snoRNA.

In one embodiment, the two or more oligonucleotides form a single guide region corresponding to one of the two hairpin structures of the wild type H/ACA snoRNA, preferably the hairpin structure at the 3′ terminal part of the wild type H/ACA snoRNA, more preferably wherein the 5′ terminal nucleotide corresponds to a nucleotide from a region between the two hairpin structures of the wild type H/ACA snoRNA.

In one embodiment, at least one of the two or more partially duplexed oligonucleotides consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

In one embodiment, the partially duplexed oligonucleotide has an equivalent length of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

In one embodiment, the partially duplexed nucleic acid molecule comprises one or more nucleosides and/or inter-nucleosidic linkages that are non-naturally modified.

In one embodiment, the non-natural modification comprises a modified sugar moiety. In one embodiment, the modified sugar moiety is 2′-deoxy, 2′-O-alkyl, 2′-halogen, or 2′-O-methoxyethyl (2′-MOE).

In one embodiment, the one or more non-natural inter-nucleosidic linkages is a phosphodiester linkage, a phosphorothioate (PS) linkage, a phosphorodithioate linkage, a phosphotriester linkage, or a methylphosphonate (MP) linkage, or any combination thereof.

In one embodiment, at least one of the two or more oligonucleotides is positioned in an intron sequence from which it is expressed. In one embodiment, the intron sequence is located between an upstream exon A sequence and a downstream exon B sequence. In one embodiment, the exon A/intron/exon B sequence is present in a vector, preferably a plasmid or a viral vector. In one embodiment, the exon A sequence is exon 1 of the human β-globin gene, and the exon B sequence is exon 2 of the human β-globin gene.

In one embodiment, at least one of the two or more oligonucleotides is present in a vector, such as a plasmid, and wherein at least one of the two or more oligonucleotides is transcribed from a CMV or a pol-III promoter, preferably a U6 or an H1 promoter.

In one embodiment, the target RNA comprises a mutation that is associated with a genetic disorder. In one embodiment, the mutation results in a Premature Termination Codon (PTC), wherein the PTC is the cause of the genetic disorder. In one embodiment, the target uridine is in the PTC.

In one embodiment, the composition is for use in the treatment, prevention, delay or amelioration of Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase deficiency, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, or cancer.

In some embodiments, the invention relates to a method for converting a uridine in a target RNA molecule into a pseudouridine, comprising the steps of contacting a target RNA comprising a target uridine with a composition comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme, in the presence of a pseudouridylation enzyme or RNP complex and allowing the uridine to be converted thereby.

In some embodiments, the method comprises administering a composition comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme to a mammalian cell comprising a target RNA comprising a target uridine.

In some embodiments, the two or more oligonucleotides are combined prior to being administered to the mammalian cell.

In some embodiments, the pseudouridylation enzyme or RNP complex is naturally present in the mammalian cell.

In some embodiments, the invention relates to a composition comprising one or more vectors, wherein the one or more vectors encode two or more oligonucleotides, and wherein each of the one or more vectors comprise an intron sequence that is located between an upstream exon A sequence and a downstream exon B sequence, wherein the exon A sequence and the exon B sequence are of a gene that is not the natural gene for the intron sequence, and wherein the intron sequence of each of the one or more vectors encode at least one of the two or more oligonucleotides, and wherein the two or more oligonucleotides associate to form a composition comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme.

In some embodiments, the composition comprises a single vector, and the intron of the single vector encodes the two or more oligonucleotides.

In some embodiments, the composition comprises two or more vectors, and each vector comprises an intron encoding one of the two or more oligonucleotides.

In some embodiments, the exon A sequence is exon 1 of the human β-globin gene, and the exon B sequence is exon 2 of the human s-globin gene.

In some embodiments, the vector is a plasmid or a viral vector.

In some embodiments, the composition is for use in the treatment, prevention or amelioration of Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase deficiency, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, or cancer.

In some embodiments, the invention relates to a pharmaceutical composition comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme, and one or more of a pharmaceutically acceptable carrier, stabilizer or solvent.

In some embodiments, the invention relates to a pharmaceutical composition comprising one or more vectors, wherein the one or more vectors encode two or more oligonucleotides, and wherein each of the one or more vectors comprise an intron sequence that is located between an upstream exon A sequence and a downstream exon B sequence, wherein the exon A sequence and the exon B sequence are of a gene that is not the natural gene for the intron sequence, and wherein the intron sequence of each of the one or more vectors encode at least one of the two or more oligonucleotides, and wherein the two or more oligonucleotides associate to form a composition comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme, and one or more of a pharmaceutically acceptable carrier, stabilizer or solvent.

In some embodiments, the invention relates to a method for converting a uridine in a target RNA molecule into a pseudouridine in a cell, preferably a human cell, comprising the steps of:
administering to the cell a composition comprising one or more vectors, wherein the one or more vectors encode two or more oligonucleotides, and wherein each of the one or more vectors comprise an intron sequence that is located between an upstream exon A sequence and a downstream exon B sequence, wherein the exon A sequence and the exon B sequence are of a gene that is not the natural gene for the intron sequence, and wherein the intron sequence of each of the one or more vectors encode at least one of the two or more oligonucleotides, and wherein the two or more oligonucleotides associate to form a composition comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme;
allowing the transcription of the one or more exon A/intron/exon B sequence;
allowing splicing and the formation of the two or more oligonucleotides positioned in the intron;
allowing the two or more oligonucleotides to associate to form a partially double stranded nucleic acid molecule; and
allowing the partially double stranded nucleic acid molecule to form a complex with the target RNA molecule,
wherein the complex is capable of engaging a mammalian pseudouridylation enzyme to form a functional RNP complex, wherein the snoRNA correctly positions the target uridine for it to be converted by the RNP complex, and wherein the target uridine is converted by the RNP complex to a pseudouridine.

In some embodiments, the invention relates to a mammalian cell comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme.

In some embodiments, the invention relates to a mammalian cell comprising one or more vectors, wherein the one or more vectors encode two or more oligonucleotides, and wherein each of the one or more vectors comprise an intron sequence that is located between an upstream exon A sequence and a downstream exon B sequence, wherein the exon A sequence and the exon B sequence are of a gene that is not the natural gene for the intron sequence, and wherein the intron sequence of each of the one or more vectors encode at least one of the two or more oligonucleotides, and wherein the two or more oligonucleotides associate to form a composition comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme.

### BRIEF DESCRIPTION OF THE DRAWINGS

FIG. 1 shows the uridine and the pseudouridine (ψ) structure. The hydrogen bond acceptor (a) and hydrogen bond donor (d) sites are shown.

FIG. 2 shows the list of residues participating in the guide RNA attachment. Single-letter amino acid code is followed by the residue number reported in the H/ACA box RNP particle structure (PDB ID 2HVY). Atoms potentially involved in hydrogen bonds formation are indicated after the residue number. The amino-acids of the archaea species Pyrococcus furiosus (P. furiosus) H/ACA box RNP possessing the ability to generate hydrogen bond contacts with the RNA oxygen-phosphate backbone and bases are reported on the left and the right panel, respectively. Empty circles represent potential hydrogen bonds to the RNA sugar-phosphate backbone whereas filled circles indicate potential hydrogen bonds to the RNA bases. Residues belonging to the Cbf5 homologue protein, Nop10 and L7ae are indicated with asterisks, ampersands and hashes, respectively. The sequence of the human-derived guide region, as shown here in both left and right panel is: 5′-GGGUCCGCCUUGAGUGCCCGGGUGAGAAGCAUGAUCCCGGGU AAUUAUGGCGGACCCACAG-3′ (SEQ ID NO:4).

FIG. 3 shows the positions within the H/ACA P. furiosus snoRNA that were examined relative to the preservation of the hydrogen-bonds interaction network. The sequence is the same as in FIG. 2. Empty circles (left panel) and filled circles (right panel) specify the RNA backbone positions where PS linkages and 2′-OMe modifications are tolerated, respectively (without disrupting potential intermolecular connections). The open circles refer to the tolerated PS linkage on the 3′ side of the nucleotide that is next to the circle.

FIG. 4, comprising FIG. 4A and FIG. 4B, shows the basic structure of human H/ACA ACA19 snoRNA (FIG. 4A) bound to its natural target sequences in the 28S rRNA, with the target nucleotide shown in its pseudouridylated form (4)). The snoRNA comprises two hairpin structures within which the internal target-binding loops are located, as well as the H box between the hairpins and the ACA box at the 3′-end. The sequence of this full length wild type ACA19 snoRNA with the H-box underlined (boxed in the figure) and the ACA box in bold (also boxed in the figure) is: 5′-GUGCACAUUUCAUUGACCUGCUUUCUUUUAUGUGAGUAGUGUUAUUUCUUAUGUGC UAUACAAAUAAUUGAAGGCUAAUUAGCAGUAUAACUAUAAAUAGUAAUGCUGCCUGU GUCCUUCAGACAAAA-3′ (SEQ ID NO:5).

The two 28s rRNA sequences are as follows: 5′-aaagugaagaaauucaaugaagcgcggg-3′ (SEQ ID NO:6; U3709; left guide region), and 5′-gaauccgacuguuuaauuaaaaca-3′ (SEQ ID NO:7; U3618; right guide region). In both 28s rRNAs the uridine that is converted to pseudo-U is underlined.

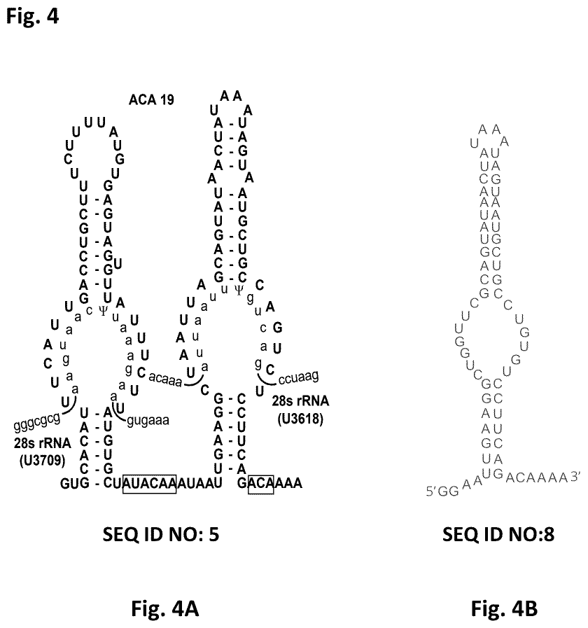

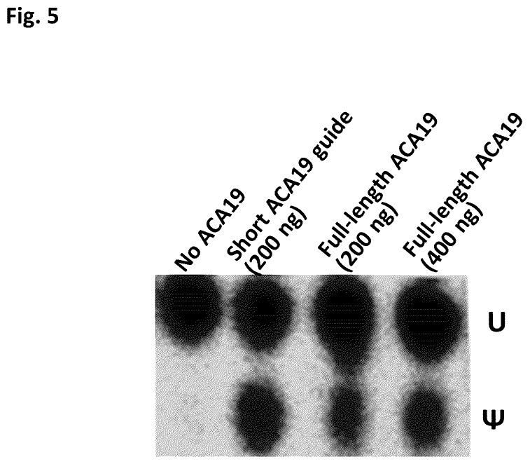

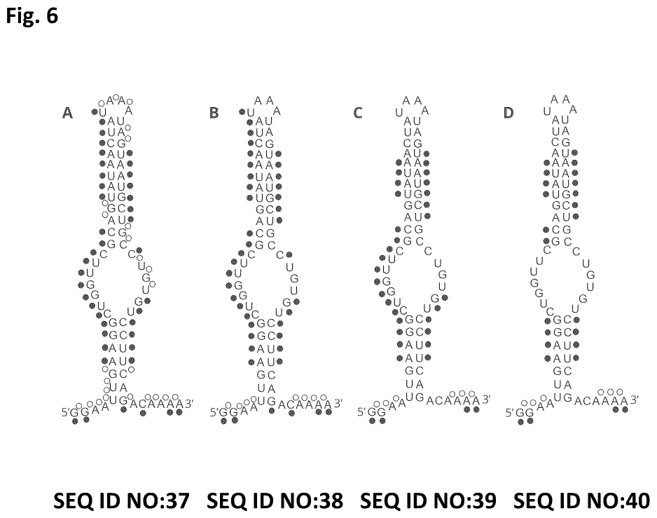

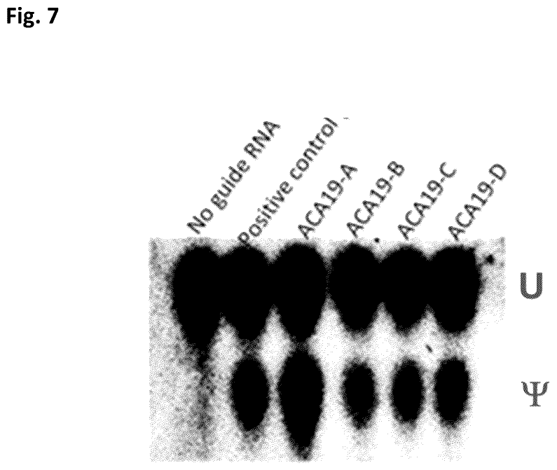

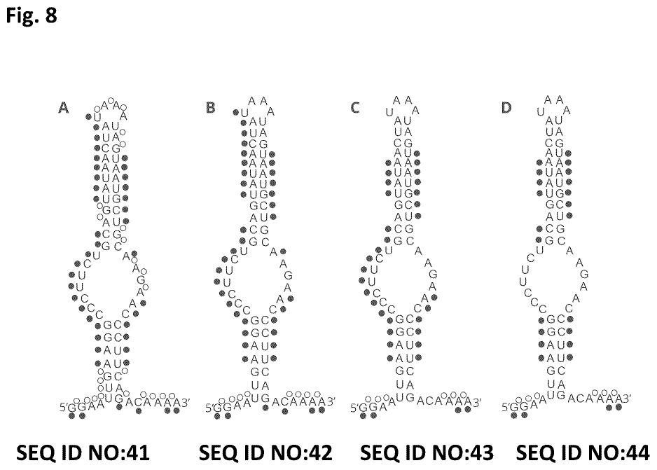

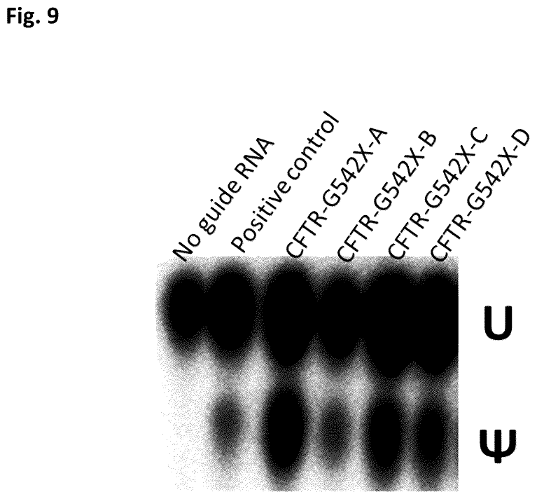

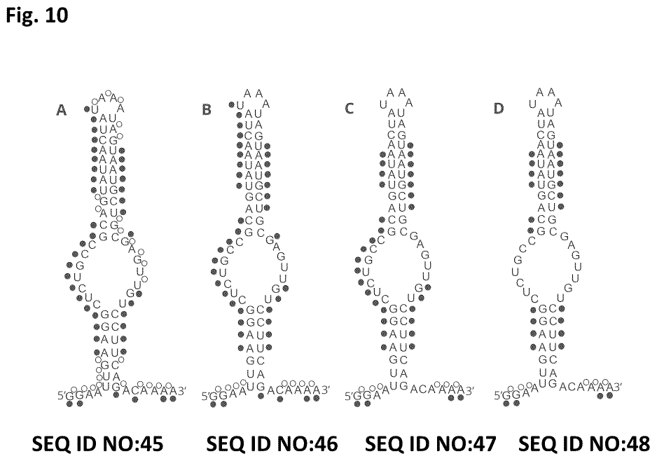

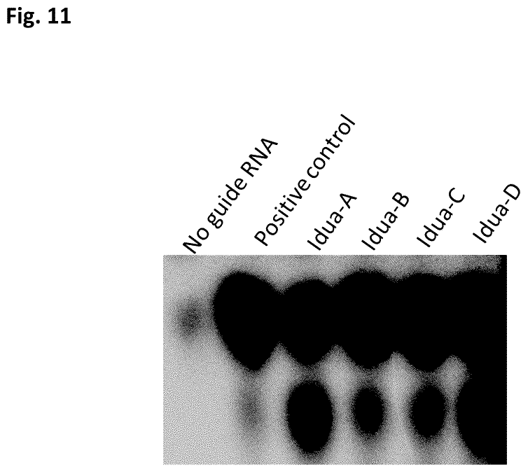

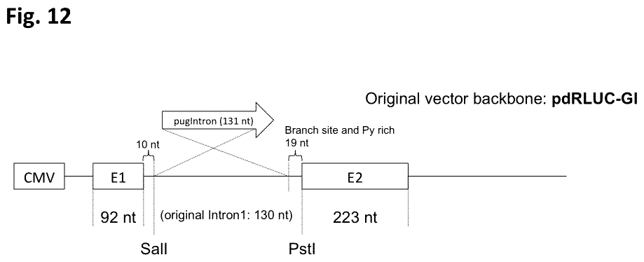

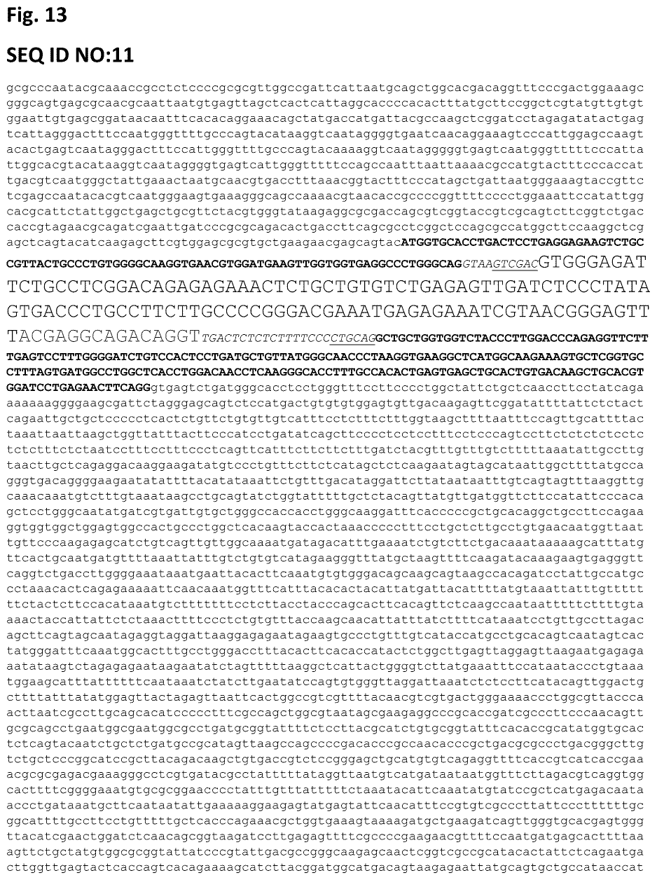

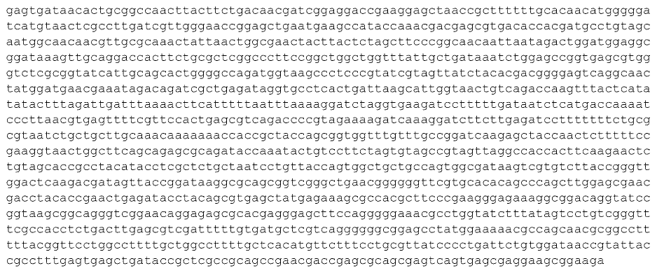

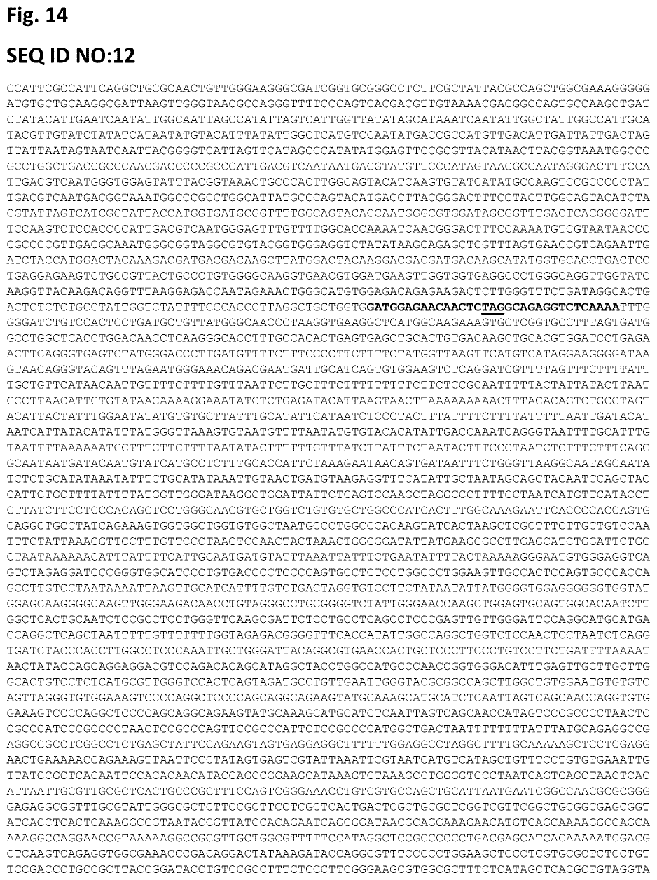

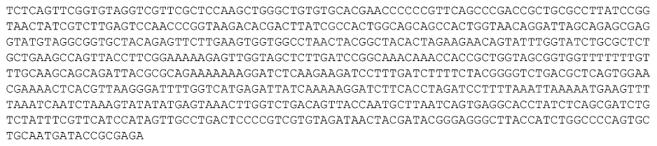

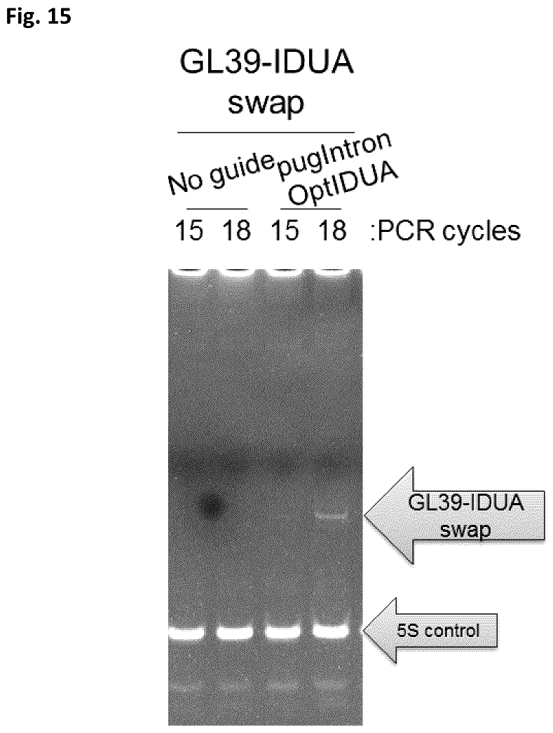

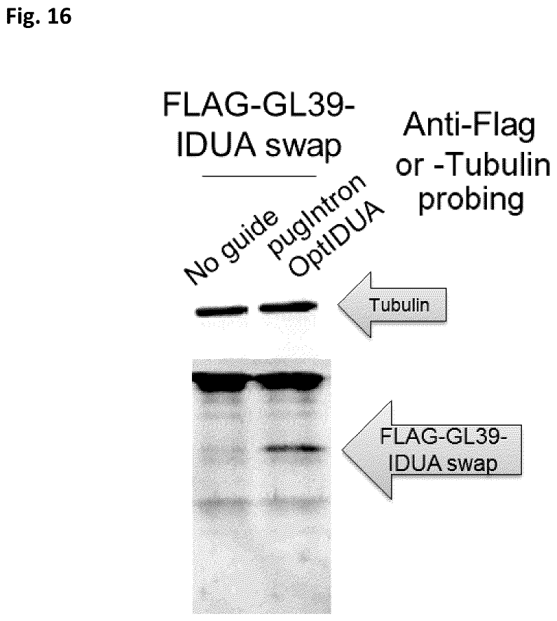

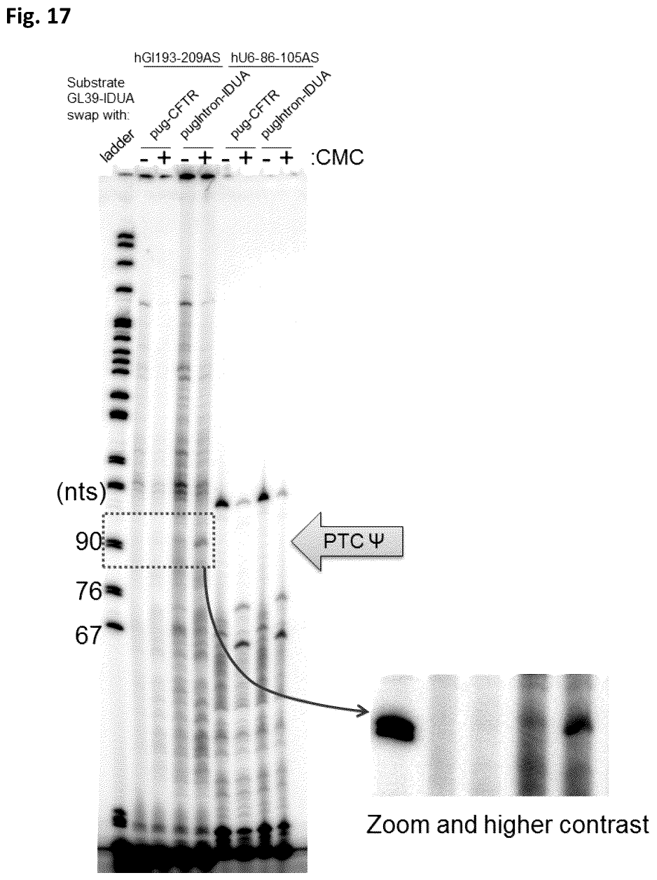

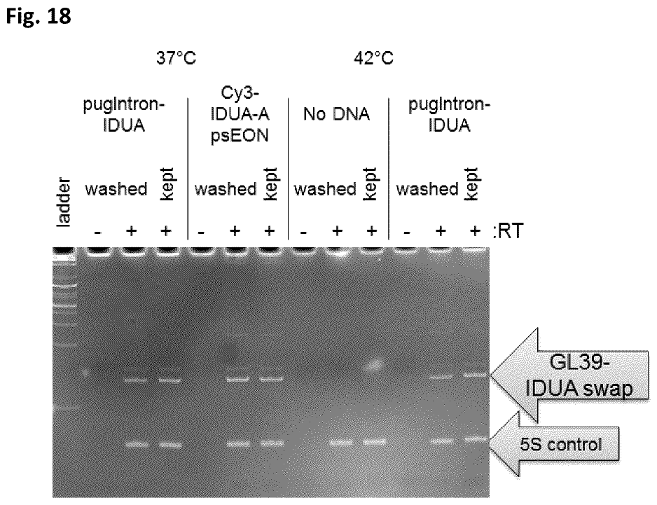

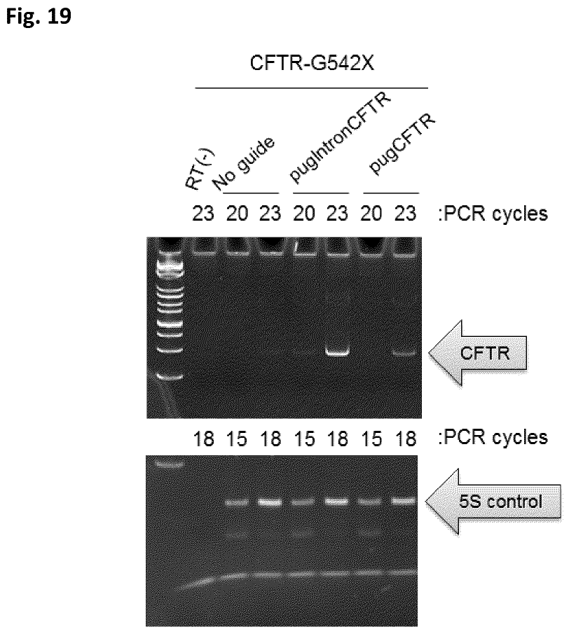

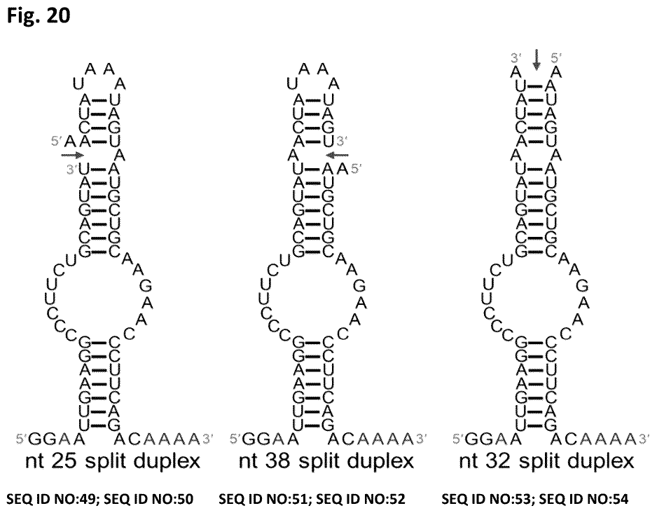

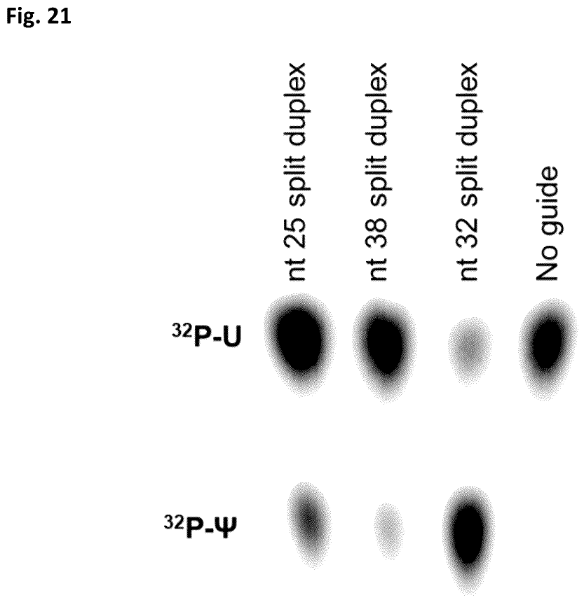

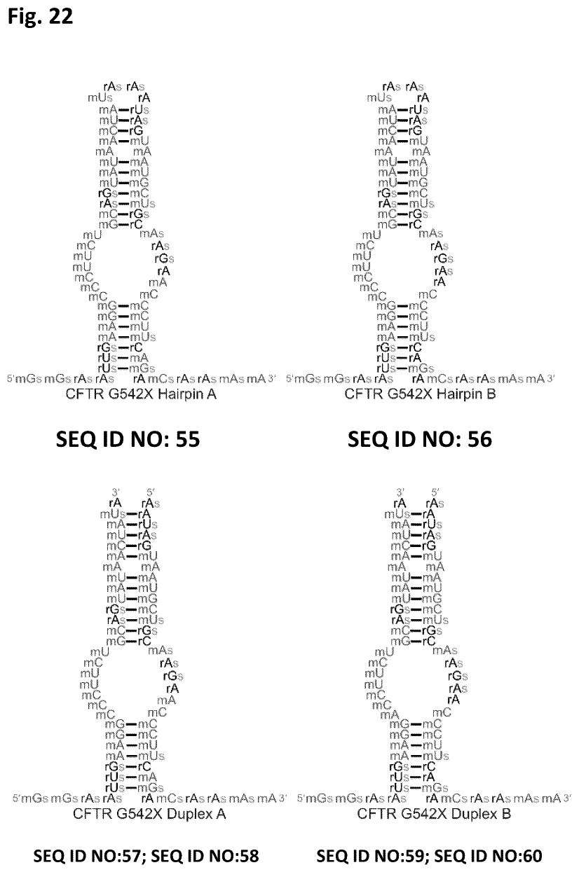

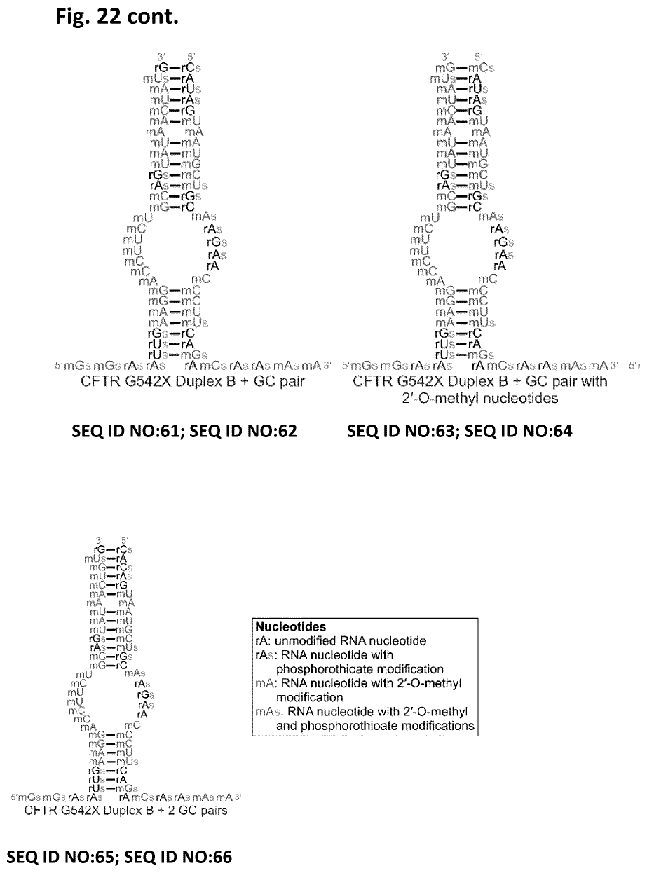

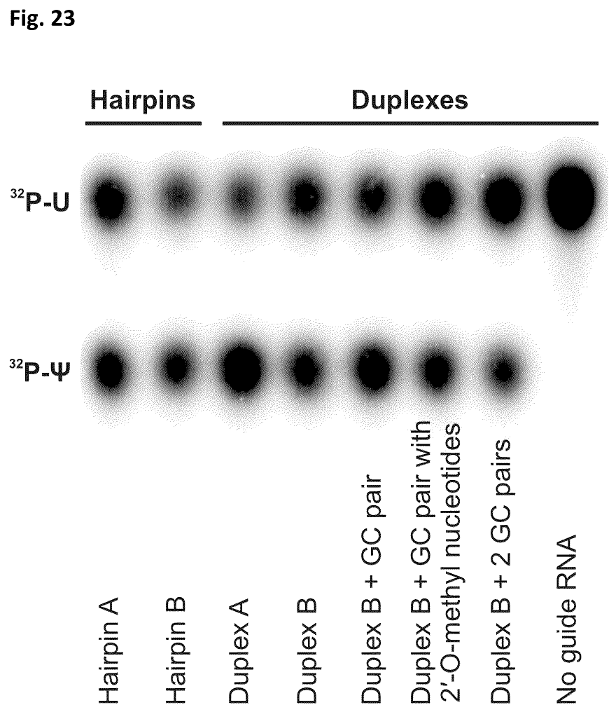

The basic structure of the shortened pseudouridylation guide RNA as described herein is given on the right (FIG. 4B) and has the following sequence, which differs at the 5′-end because of residual nucleotides from the transcription, and which differs in the target loop because the sequence of the target mRNA differs from the 28s rRNA sequence (SEQ ID NO:7): 5′-GGAAUUGAAGGCU GGUUCGCAGUAUAACUAUAAAUAGUAAUGCUGCCUGUGUCCUUCAGACAAAA-3′ (SEQ ID NO:8).

FIG. 5 shows results from a pseudouridylation assay of a radioactively labelled substrate RNA using either no guide (as the negative control), the shortened pseudouridylation guide RNA or the full-length ACA19 snoRNA at the amount indicated. U and W indicate the migration of uridine and pseudouridine in the thin layer chromatography, respectively.

FIG. 6 shows the four ACA19 pseudouridylating editing oligonucleotides (psEONs, SEQ ID NOS: 37-40), and the positions of the chemical modifications that were introduced: black dots indicate 2′-OMe modifications on the ribose moiety of that nucleotide, and open dots indicate PS modifications of the inter-nucleosidic linkage.

FIG. 7 shows the result of pseudouridylation using the four ACA19 psEONs together with the corresponding guide RNA (lacking chemical modifications) as a positive control and a negative control (no guide RNA). U and 4 indicate the migration of uridine and pseudouridine in the thin layer chromatography, respectively.

FIG. 8 shows the four CFTR-G542X psEONs (SEQ ID NOS: 41-44, the guide pocket sequence is changed to target CFTR substrate in the same ACA19 backbone) and the positions of the chemical modifications that were introduced: dark dots indicate 2′-OMe modifications on the ribose moiety of that nucleotide, and open dots indicate phosphorothioate modification of the inter-nucleosidic linkage. The unmodified sequence for all these four psEONs is: 5′-GGAAUUGAAGGCCCUUCUGCAGUAUAACUAUAAAUAGUAAUGCUGCAAGAACCCUUC AGACAAAA-3′ (SEQ ID NO:9).

FIG. 9 shows the result of pseudouridylation using the four CFTR-G542X psEONs in parallel with the corresponding guide RNA (lacking chemical modifications) as a positive control and no guide RNA as a negative control. U and P indicate the migration of uridine and pseudouridine in the thin layer chromatography, respectively.

FIG. 10 shows the four/dua-W392X psEONs (from left to right Idua-A, —B, —C and -D, SEQ ID NOS: 45-48) and the positions of the chemical modifications that were introduced: black dots indicate 2′-OMe modifications on the ribose moiety of that nucleotide, and open dots indicate PS modification of the inter-nucleosidic linkage. The unmodified sequence for all these four psEONs is: 5′-GGAAUUGA AGGCUCUGCCGCAGUAUAACUAUAAAUAGUAAUGCUGCGAGUUGUCCUUCAGACAAA A-3′ (SEQ ID NO:10).

FIG. 11 shows the result of pseudouridylation using the four/dua-W392X psEONs (Idua-A, -B, -C and -D) in parallel with the corresponding guide RNA (lacking chemical modifications) as a positive control and no guide RNA as a negative control. U and 4P indicate the migration of uridine and pseudouridine in the thin layer chromatography, respectively.

FIG. 12 shows a schematic view of the puglntron-IDUA construct displaying the introduction of the 131 nt pugintron insert located between the SalI site on the 5′ end and the PstI site on the 3′ end, with the 92 nt exon 1 (E1) and the 223 nt exon 2 (E2) of the human β-globin gene up- and downstream thereof, respectively. Transcription is driven by a CMV promoter.

FIG. 13 shows the entire nucleotide sequence of the puglntron-mIDUA plasmid (SEQ ID NO:11) as shown schematically in FIG. 12, carrying the to-be-spliced out guide RNA. The plasmid sequences are given in lower case. The 131 nt guide RNA is given in a large font.

The guide RNA is spliced out together with 10 nt upstream and 23 nt downstream. The upstream 92 nt exon 1 (E1 in FIG. 12) and the downstream 223 nt exon 2 (E2 in FIG. 12) are given in upper case and bold. The SalI and PstI restriction enzyme recognition sites are underlined.

FIG. 14 shows the entire sequence of the target pFLAG-GL39-IDUA swap expression vector (SEQ ID NO:12; originated from pFLAG2CMV2-HBB). The 33 nt mIDUA PTC region is in bold, and the TAG premature termination codon is underlined.

FIG. 15 shows the RT PCR analysis of β-globin mRNAs from HEK293 cells transfected with substrate plasmid “GL39-IDUA swap with or without pugIntronOpt-IDUA. The GL39 arrow indicates the product observed after RT-PCR. Lane 1 and 3 represent 15 PCR cycles and lane 2 and 4 represent 18 PCR cycles. Globin mRNA level was normalized by 5S control (lower arrow) and was found to be 0.00437 in lane 2, and 0.1617 in lane 4. Guide-induced RNA modification (here: pseudouridylation) suppressed NMD and upregulated the intact mRNA level 37-fold.

FIG. 16 shows the western blot analysis of flag-tagged globin proteins from HEK293 cells transfected with substrate FLAG-GL39-IDUA swap plasmid and guide pug-Intron-IDUA plasmid. Cells were transfected with 250 ng substrate and 2.5 μg guide using PEI at 100% confluency.

FIG. 17 shows the results of a primer extension experiments using total RNA obtained from HEK293T cells transfected with the substrate GL39-IDUA swap plasmid with puglntron-IDUA as the guide RNA expressing plasmid or pug-CFTR as the negative control plasmid. RNA was treated with CMC and primer extension was carried out with a globin-specific primer and with a U6 specific primer as a control. The position of the 92 nt product terminated at the ψ-CMC residue is given by an arrow. This area is again represented in the zoomed-in panel. GL39+72 is the hG1193-209AS globin-specific primer. 90, 76 and 67 are size references from a pBR322-MspI digest (NEB #N3032).

FIG. 18 shows the RT-PCR products from total RNA obtained from cells transfected with the GL39-IDUA swap substrate plasmid and the puglntron-IDUA guide RNA expressing plasmid or with the Cy3-IDUA-A psEON (wherein the guide RNA is not intronically embedded). The GL39-IDUA arrow indicates the product observed after RT-PCR. The 5S control is also given with an arrow. No difference was observed when cells were washed (wash), or when the medium was kept (kept) on the cells after transfection. RT means reverse transcriptase (+yes, or −no).

FIG. 19 shows the RT-PCR analysis of CFTR mRNAs from HEK293 cells transfected with substrate plasmid carrying the CFTR-G542X mutation, with either no guide (lanes 2 and 3 right from the ladder), negative control pugCFTR (lanes 6 and 7 right from the ladder) and guide snoRNA carrying plasmid pugIntCFTR (lanes 4 and 5 right from the ladder). The arrow indicates the product observed after RT-PCR. Lane 1 after the ladder represents the PCR without RT. Lanes 2, 4 and 6 represent product after 20 PCR cycles, while lanes 1, 3, 5, and 7 represent the product obtained after 23 cycles. The lower panel shows the 5S control after respectively 15 and 18 PCR cycles (instead of 20 and 23). The presence of the guide RNA expressed from pugIntCFTR clearly leads to NMD suppression as a stronger PCR product signal is observed.

FIG. 20 shows a schematic for the RNA psEONs that form the three different ACA19-CFTR-G542X guide duplexes (SEQ ID NOS: 49 and 50, SEQ ID Nos: 51 and 52, and SEQ ID Nos: 53 and 54, ACA19 backbone, with the guide pocket sequence changed to target CFTR substrate in the same). An arrow indicates the “gap” in the guide duplex, i.e. the position between the 3′ end of the oligonucleotide forming the 5′-most part of the guide and the 5′ end of the oligonucleotide forming the 3′-most part of the guide.

FIG. 21 shows the result of pseudouridylation using the duplex psEONs and a sample with no guide RNA as a negative control. U and 4) indicate the migration of uridine and pseudouridine in the thin layer chromatography, respectively.

FIG. 22 shows two full-length ACA19-CFTR-G542X pseudouridylating editing oligonucleotides (psEONs, SEQ ID NOS: 55-56) and the five duplexes formed from shorter psEONs (SEQ ID NOS:57 and 58, SEQ ID NOS: 59 and 60, SEQ ID NOS: 61 and 62, SEQ ID NOS: 63 and 64, and SEQ ID NOS: 55 and 66). The psEONs vary in their chemical compositions, and positions of the chemical modifications are indicated as follows: black uppercase letters indicate nucleotides without modifications to their ribose moieties, green lower-case letters (a, c, g and u) indicate 2′-OMe modifications on the ribose moiety of that nucleotide, and an orange lowercase letter s indicates PS modification of the linkage between the two riboses. The duplex psEONs also vary in their base composition, such that the duplexes have varying levels of internal base-pairing interactions.

FIG. 23 shows the result of pseudouridylation using the five duplex psEONs in parallel with the corresponding full-length hairpin (HP) psEONs as a positive control and no guide RNA as a negative control. U and 4P indicate the migration of uridine and pseudouridine in the thin layer chromatography, respectively.

### DETAILED DESCRIPTION OF THE INVENTION

It is known in the art that full length H/ACA RNAs (snoRNAs) can be used to site-specifically convert a target uridine to pseudouridine (ψ). By changing the nucleic acid sequence of the two major loops that serve as guide for the natural substrate RNA, it was shown that other sequences than the natural sequence can be pseudouridylated in yeast and Xenopus systems. Further, smaller pseudouridylation guide RNAs have been previously generated that are still be catalytically active. Exemplary small pseudouridylating editing oligonucleotides, or psEONs, are described in detail in WO2019191232A2 which is incorporated herein by reference in its entirety. Such guides have utility in the treatment of any genetic disorder in which a uridine (which may represent a mutation) would be converted to a ψ, and thereby either influence the content of the translated protein, or influence splicing, or increase (or decrease) protein translation from the resulting mRNA. However, there remains a need for improving the potency and pharmacokinetic properties of pseudouridylation guides. The present invention relates to the generation of short oligonucleotides that can complex to form a pseudouridylation guide compound (also referred to herein as “split pseudouridylating editing oligonucleotides”, or “split psEONs”) that are individually shorter (in respect of nucleotide length) than the pseudouridylation guide molecules from the art, thus reducing synthesis cost, but that can complex together to form a catalytically active pseudouridylation guide molecule. In some embodiments, the invention provides a combination of two or more oligonucleotides that complex to form a split psEON. a nucleic acid sequence is provided that is or comprises a guide for targeted pseudouridylation. In some embodiments, the invention provides a combination of two or more oligonucleotides, wherein each oligonucleotides is expressed as a pol-III expression construct, using a pol-III promoter such as U6 or H1. In some embodiments, the invention provides a combination of two or more expression constructs, e.g., a combination of plasmid or viral vectors, such as an AAV vectors or the like expressing the combination of two or more oligonucleotides that complex to form a split psEON.

The originally identified (‘full length’) H/ACA RNAs have a median length of 133 nucleotides forming an evolutionary conserved hairpin-hinge-hairpin-tail secondary structure that carries a consensus ANANNA sequence in the hinge region (H box) and an ACA triplet exactly three positions from the 3′-end (ACA box). Identification of target uridines is achieved by two 3-10 nucleotide-long antisense elements in the bulge of one or both hairpins (pseudouridylation pocket) directly 5′ and 3′ of the upper stem. These two elements hybridize to sequences immediately 5′ and one nucleotide 3′ of the target uridine thereby framing it. This positions the target uridine 14-15 nucleotides from the H or ACA box.

In one embodiment, the split psEON of the invention comprises only one pseudouridylation pocket (as opposed to two such structures typically found in natural H/ACA RNA structures and the artificial snoRNAs described in the art).

In some embodiments, the oligonucleotides of the invention comprise a combination of a first oligonucleotide and second oligonucleotide, that when complexed form a secondary structure comprising a duplex region-pseudouridylation pocket-duplex region. In some embodiments, at least one of the duplex regions comprises a hairpin structure. In some embodiments, the first oligonucleotide comprises a 3-10 nucleotide-long antisense element and the second oligonucleotide comprises a 3-10 nucleotide-long antisense element, such that when the two or more oligonucleotides form a complex through base-pairing in the duplex regions, the 3-10 nucleotide-long antisense elements of the first and second oligonucleotides form the pseudouridylation pocket, comprising a guide region. In some embodiments, the first oligonucleotide comprises the 5′ domain of the guide region and the second oligonucleotide comprises the 3′ domain of the guide region, such that when the two or more oligonucleotides form a complex through base-pairing in the duplex regions, the 5′ domain of the guide region and 3′ domain of the guide region form the full guide region in the pseudouridylation pocket. Therefore, in some embodiments, the composition comprises a split psEON comprising a first oligonucleotide comprising the 5′ end of the split psEON a first hybridization domain (hyb-A domain), a pseudouridylation pocket domain, comprising the 5′ domain of the guide region, and a second hybridization domain (hyb-B domain); and a second oligonucleotide comprising a hybridization domain (hyb-B domain), a pseudouridylation pocket domain, comprising the 3′ domain of the guide region, a second hybridization domain (hyb-A domain), and the 3′ end of the split psEON, wherein the first and second oligonucleotides hyb-A domains hybridize with each other and the first and second oligonucleotides hyb-B domains hybridize with each other. In some embodiments, each of the hyb-A and hyb-B domains of the split psEON comprise 3-20 nucleotides. In some embodiments, the pseudouridylation pocket domain comprises 3-20 nucleotides. In some embodiments, the pseudouridylation pocket domain of the first and second oligonucleotide molecules does not hybridize, but remains single-stranded, forming a bubble.

In some embodiments, the split psEON comprises SEQ ID NO: 49 and SEQ ID NO:50. In some embodiments, the split psEON comprises SEQ ID NO:51 and SEQ ID NO:52. In some embodiments, the split psEON comprises SEQ ID NO:53 and SEQ ID NO:54.

In some embodiments, one or more of the oligonucleotides that complex to form a split psEON of the present invention comprise a chemical modification. Exemplary chemical modification that can be incorporated into one or more of the oligonucleotides that complex to form a split psEON of the present invention include, but are not limited to, 2′-deoxy, 2′-O-alkyl, 2′-halogen, 2′-O-methoxyethyl (2′-MOE), 2′-O-[2-(methylamino)-2-oxoethyl](2′-NMA), 2′-O-methyl (2′-OMe), 2′-deoxy-2′-fluoronucleoside, 2′-fluoro-β-D-arabinonucleoside, Locked Nucleic Acid (LNA), constrained ethyl 2 ′-4′-bridged nucleic acid (cEt), S-cEt, Hexitol Nucleic Acid (HNA), a bicyclic sugar moiety, and a tricyclic sugar moiety.

In some embodiments, one or more of the oligonucleotides that complex to form a split psEON of the present invention comprise one or more non-natural inter-nucleosidic linkages. Exemplary non-natural inter-nucleosidic linkages include, but are not limited to phosphodiester linkages, phosphorothioate (PS) linkages, alkylphosphate linkages, alkylphosphonate linkages, 3-methoxypropylphosphonate linkages, phosphorodithioate linkages, phosphotriester linkages, methylphosphonate linkages, aminoalkyl phosphotriester linkages, alkylenephosphonate linkages, phosphinate linkages, phosphoramidate linkages, phosphorothioamidate linkages, phosphorodiamidate linkages (e.g., including Phosphorodiamidate Morpholino (PMO), 3 ′aminoribose or 5′ aminoribose) linkages, aminoalkyl phosphoramidate linkages, phosphorothioamidate linkages, thioalkyl phosphonate linkages, thioalkyl phosphotriester linkages, phosphoroselenoate linkages, and phosphoroborate linkages, or any combination thereof. The present invention relates to a combination of nucleic acid molecules, capable of forming a complex with a target RNA molecule in a system (e.g. a cellular extract, a cell, a tissue or organ (e.g. on a chip), a living organism, preferably of mammalian origin), and capable of forming a functional (i.e. capable of pseudouridylation) RNA protein (RNP) complex, wherein the target RNA molecule comprises a target uridine for conversion into ψ, wherein the combination of oligonucleotides forms a split psEON prior to, or concurrent with, forming a complex with the target RNA molecule. In some embodiments, one or more of the combination of oligonucleotides comprises a (non-natural) chemical modification of one or more nucleotides or inter-nucleosidic linkages.

In some embodiments, the split psEON comprises SEQ ID NO: 57 and SEQ ID NO:58. In some embodiments, the split psEON comprises SEQ ID NO:59 and SEQ ID NO:60. In some embodiments, the split psEON comprises SEQ ID NO:61 and SEQ ID NO:62. In some embodiments, the split psEON comprises SEQ ID NO:63 and SEQ ID NO:64. In some embodiments, the split psEON comprises SEQ ID NO:65 and SEQ ID NO:66.

In some embodiments, the oligonucleotides of the invention which complex to form a split psEON according to the invention are synthetic oligonucleotides. In some embodiments, the psEONs according to the invention are oligoribonucleotides (full RNA) but may comprise DNA. In some embodiments, the oligonucleotides that form the split psEONs may be expressed in situ, e.g. from a plasmid or a viral vector. In addition, in some embodiments, the oligonucleotides that form the split psEONs may be a mix of biologically expressed components and synthetic components, such as tags or linkers. In some embodiments, the oligonucleotides that form the split psEONs may be used as such (‘naked’), or conjugated to other components, such as ligands for targeting, for uptake and/or for intracellular trafficking. In some embodiments, the oligonucleotides that form the split psEONs may be used in aqueous solutions (generally pharmaceutically acceptable carriers and/or solvents), or formulated using transfection agents, liposomes or nanoparticulate forms (e.g., SNALPs, LNPs and the like). Such formulations may comprise functional ligands to enhance bioavailability and the like.

Among the rich variety of non-coding RNAs, small nucleolar RNAs are divided in two categories: the box C/D and the box H/ACA snoRNAs. Both contribute to the modification of ribosomal RNAs after transcription. Using endogenous RNP enzymes, synthetic oligonucleotides can potentially mimic the guiding H/ACA snoRNAs and bind to RNA targets allowing the conversion of uridine to ψ in a site-specific manner. In order to convert uridine to ψ in RNA targets, chemically modified oligonucleotides resembling H/ACA snoRNAs can recruit endogenous RNP enzymes. To design functional split psEONs, experimental data is provided that demonstrates the generation of split psEONs with the split at different positions of the duplex. The functionality was checked with in vitro enzymatic assays (as described below).

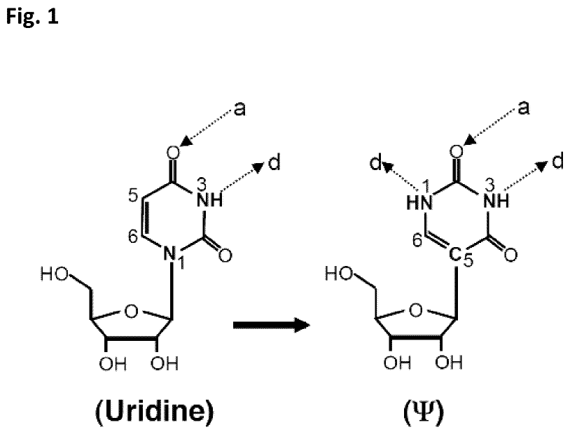

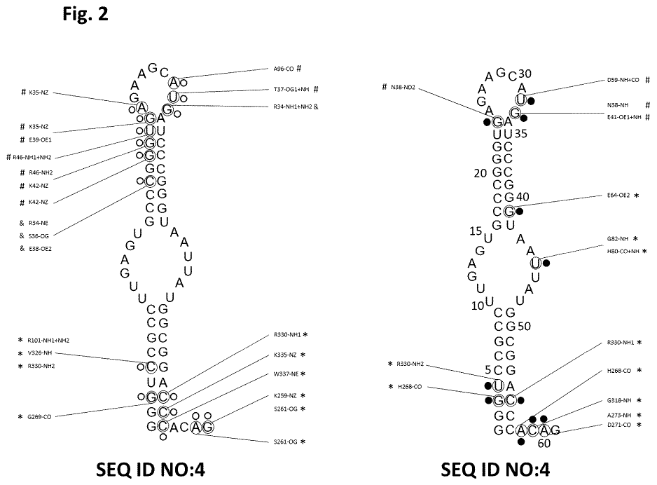

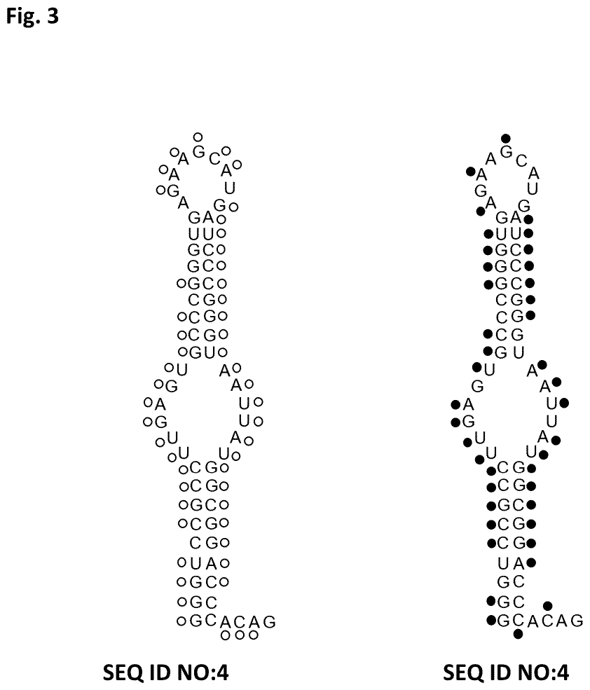

The split psEON design is based, in part, on the design of psEONs which is described in detail in WO2019191232A2 and herein described in Examples 1-5 and FIGS. 1-19. The split psEONs of the invention have the advantage of, taken separately, being shorter oligonucleotides than the psEONs, but having the same functionality (pseudouridylation of a target uridine in a target RNA in a mammalian cell.)

In some embodiments, the invention relates to a combination of oligonucleotide molecules for pseudouridylation of a target uridine in a target RNA in a mammalian cell, wherein the combination of oligonucleotide molecules form a partially double stranded molecule comprising a duplex region-pseudouridylation pocket-duplex region structure (split psEON), wherein the pseudouridylation pocket comprises a guide region capable of forming a complex with the target RNA comprising the target uridine, wherein the split psEON:target RNA complex is capable of engaging a mammalian pseudouridylation enzyme, wherein the guide region assists in positioning the target uridine in the split psEON:target RNA complex for it to be converted to a pseudouridine by the mammalian pseudouridylation enzyme. In one embodiment, the split psEON:target RNA complex is RNA/RNA. The RNA/RNA complex is able to recruit, or to get involved, a pseudouridylation enzyme that is preferably naturally present in the mammalian cell, but may be, in another embodiment, co-introduced with the nucleic acid molecule(s) into the mammalian cell. In one embodiment, the mammalian cell in which the pseudoruridylation takes place is a human cell. In one embodiment, the pseudouridylation enzyme is part of a ribonucleoprotein (RNP) complex capable of acting on an H/ACA-snoRNA.

In one embodiment, one or more oligonucleotide that is part of a split psEON of the invention comprises one or more nucleosides and/or inter-nucleosidic linkages that are non-naturally modified. Non-naturally means that the modification is in nature not present in a wild type snoRNA. The modification is preferably introduced to render the nucleic acid molecule more stable towards breakdown by nucleases and thereby to increase efficiency.

In one embodiment, the split psEON of the invention formed by the hybridization of two or more oligonucleotides of the present invention comprises a single guide region corresponding to one of the two hairpin structures of the wild type H/ACA snoRNA. In some embodiments, the oligonucleotides of the invention that hybridize to form the split psEON of the invention are each less than about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length. In one embodiment, the one or more oligonucleotide molecules that hybridize to form a split psEON of the invention consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In some embodiments, the total length of the split psEON formed by the hybridization of two or more oligonucleotides comprises an equivalent length of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. For example, in some embodiments, the invention comprises a combination of a first oligonucleotide molecule comprising 10-50 nucleotides and a second oligonucleotide comprising 10-50 nucleotides, wherein the total length of the split psEON (i.e., the sum of the first and second oligonucleotides) comprises an equivalent length of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

In one embodiment, the invention also relates to a combination of oligonucleotide molecules according to the invention, wherein the oligonucleotide molecules are positioned in an intron sequence from which they are expressed, and wherein the intron sequence is located between an upstream exon A sequence and a downstream exon B sequence. The intron may comprise (besides the one or more oligonucleotide of the present invention for formation of a split psEON) additional nucleotides. Since one or more oligonucleotide of the invention is expressed from the intron sequence, such additional nucleotides may be selected to render the most efficient expression from the intron. In some embodiments, the exon A/intron/exon B sequence is present in a vector, preferably a plasmid or a viral vector. Such a vector can be used to deliver the exon-intron-exon sequence to the cell. Additional introns and exons may be present in such a vector. In a particularly preferred embodiment, the exon A sequence (upstream of the intron that carries one or more oligonucleotide of the present invention for formation of a split psEON (which is expressed after transcription)) comprises or consists of exon 1 of the human β-globin gene, and the exon B sequence (downstream of the intron that carries the nucleic acid encoding the nucleic acid molecule (which is expressed after transcription)) comprises or consists of exon 2 of the human β-globin gene. The skilled person knows that vectors may carry DNA or RNA, and are generally used to express the nucleic acid molecule of the present invention after the vector is processed in the cell in which it is introduced. Such is generally through transcription of the DNA or RNA present in the vector. Preferred vectors are viral vectors (that may be used to infect target cells to be treated), or plasmids, that may be introduced into the cell in a variety of ways, known to the person skilled in the art.

In one embodiment, one or more oligonucleotide of the invention may be administered in a free form (or ‘naked’, without the context of a vector), be administered as a partially duplexed molecule (as a split psEON), or being delivered to a cell by any means known in the art, including, but not limited to, liposomes, or nanoparticles, or by using iontophoresis.

In one embodiment, the guide region present in the split psEON of the present invention is capable of forming a complex with the target RNA, which comprises a mutation that is associated with a genetic disorder. A non-limiting, but preferred example of such a mutation results in a Premature Termination Codon (PTC), wherein the PTC is the cause of the genetic disorder, and wherein the target uridine is in the PTC. Converting the target uridine in such a PTC to a pseudouridine, by using the means and methods of the present invention, then results in proper read-through of the reading frame during translation, thereby providing a (partly or fully) functional full-length protein.

In one embodiment, the split psEON according to the invention is for use in the treatment, prevention, delay or amelioration of Cystic fibrosis, Hurler Syndrome, alpha-1-antitrypsin (A1AT) deficiency, Parkinson's disease, Alzheimer's disease, albinism, Amyotrophic lateral sclerosis, Asthma, Bl-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase deficiency, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, a Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, or cancer.

In yet another embodiment, the invention relates to a method for converting a uridine in a target RNA molecule into a pseudouridine, comprising the steps of contacting a target RNA comprising a target uridine with a combination of oligonucleotides that can hybridize to form a split psEON according to the invention in the presence of a pseudouridylation enzyme or RNP complex and allowing the uridine to be converted thereby. In one embodiment, the pseudouridylation enzyme or RNP complex is present in a mammalian cell. In one embodiment, the cell is a human cell. In one embodiment, the pseudouridylation enzyme or RNP complex is naturally present in the mammalian cell.

In one embodiment, the split psEON:target RNA complex is capable of engaging a mammalian pseudouridylation enzyme to form a functional RNP complex, wherein the guide region of the split psEON correctly positions the target uridine for it to be converted by the RNP complex, and wherein the target uridine is converted by the RNP complex to a pseudouridine. The invention also relates to a mammalian cell comprising a nucleic acid molecule according to the invention or a vector according to the invention. In yet another embodiment, the invention relates to the use of a nucleic acid molecule of the invention in the manufacture of a medicament for the treatment of one or more of the diseases listed herein.

The invention also relates to a pharmaceutical composition comprising a combination of nucleic acid molecules according to the invention, or a split psEON according to the invention, and one or more of a pharmaceutically acceptable carrier, stabilizer, or solvent. Suitable pharmaceutically acceptable carriers are well known to the person skilled in the art.

In one embodiment, the present invention relates to nucleic acid molecules according to the present invention for use in the treatment of cystic fibrosis (CF), and in an even further preferred embodiment, the present invention relates to nucleic acid molecules according to the invention for use in the treatment of CF wherein PTCs such as those listed in Table 1, and more preferably the G542X (UGA), W1282X (UGA), R553X (UGA), R1 162X (UGA), Y122X (UAA), W1089X, W846X, and W401X mutations are modified through pseudouridylation to amino acid encoding codons, and thereby allowing the translation to full length proteins. It has for instance been well established in the art that ψAA and ψAG codons are both translated to serine orthreonine, whereas a ψGA is translated to tyrosine or phenylalanine, instead of being seen as a stop codon (Karijolich and Yu, 2011). Hence, the pseudouridylation of PTCs to any of these ψ-containing codons will generate read-through during translation or in other words, suppress termination of the protein translation and/or the potential degradation of the mRNA by nonsense-mediated decay. Hence, in one embodiment, the present invention relates to a combination of nucleic acid molecule according to the invention, or a split psEON as outlined herein, for use in the treatment of CF, wherein the nucleic acid molecule enables the conversion of a uridine present in a PTC present in the CFTR (pre-) mRNA to a ψ, and wherein the PTC results in early translation termination that eventually causes the disease. Hence, in another embodiment the invention relates to the use of a combination of oligonucleotides or a split psEON according to the invention in the manufacture of a medicament for the treatment or prevention of a disease. In one embodiment, the disease is CF. In yet another embodiment of the invention, it relates to a method for the pseudouridylation of at least one target uridine present in a PTC in a target RNA in a cell, the method comprising the steps of providing the cell with a combination of oligonucleotides, or a split psEON, according to the invention according to the invention; allowing uptake by the cell of the combination of oligonucleotides or the split psEON according to the invention (for instance while being carried by a delivery vector); allowing annealing of split psEON according to the invention to the target RNA; allowing a guide RNA-guided RNP to pseudouridylate the target uridine in the target RNA to a ψ; and optionally identifying the presence of the ψ in the targeted RNA, preferably wherein the last step comprises assessing the presence of a functional, elongated, full length and/or wild type protein when the target uridine is located in a PTC; assessing whether splicing of the pre-mRNA was altered by the pseudouridylation; or using a functional read-out, wherein the target RNA after the pseudouridylated target molecule encodes a functional, full length, elongated and/or wild type protein. Preferably, the cell in which pseudouridylation takes place, using methods and means of the present invention, is a human cell. In one preferred embodiment, each oligonucleotide for formation of the split psEON according to the invention comprises at least 20 nucleotides and is shorter than 60 nucleotides. For example, in some embodiments, the invention comprises a combination of a first oligonucleotide molecule comprising 20-50 nucleotides and a second oligonucleotide comprising 20-50 nucleotides, wherein the total length of the split psEON (i.e., the sum of the first and second oligonucleotides) comprises an equivalent length of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

As will be readily apparent to the skilled reader, the invention allows for different nucleic acid molecules according to the invention, designed for different target uridines in one and the same or different target RNAs targets, to be combined. Different nucleic acid molecules according to the invention may be used simultaneously in a single composition or in separate compositions, or consecutively. Nucleic acid molecule complexes, such as split psEONs according to the invention, may be combined with other forms of treatment, including other forms of oligonucleotide treatment. The examples provided herein serve to illustrate the invention and are by no means to be interpreted as limiting the invention in any way.

TABLE 1
List of PTC mutations in human CFTR that can be
targeted for pseudouridylation using a nucleic
acid molecule according to the present invention.

| Name of | |
|---|---|
| mutation | Mutation |
| G542X | G > T |
| W1282X | G > A |
| R553X | C > T |
| R1162X | C > T |
| Q493X | C > T |
| E60X | G > T |
| Y1092X | C > A |
| R1158X | C > T |
| Y122X | T > A |
| W1089X | G > A |
| R75X | C > T |
| W846X | G > A |
| E585X | G > T |
| Q220X | C > T |
| K710X | A > T |
| E822X | G > T |
| Q552X | C > T |
| E92X | G > T |
| Q39X | C > T |
| E831X | G > T |
| Q1313X | C > T |
| R709X | C > T |
| S466X | C > A |
| S489X | C > A |
| Q890X | C > T |
| R764X | C > T |
| S1196X | C > G |
| W401X | G > A |
| L732X | T > G |
| Q98X | C > T |
| R851X | C > T |
| W1204X | G > A |
| G330X | G > T |
| E1104X | G > T |
| Q525X | C > T |
| R785X | C > T |
| R792X | C > T |
| S912X | C > A |
| C276X | C > A |
| Q414X | C > T |
| S1255X | C > A |
| Y849X | C > A |
| E1371X | G > T |
| Y913X | T > A |
| Q1412X | C > T |
| L1254X | T > G |
| E193X | G > T |
| S4X | C > A |
| G673X | G > T |
| G27X | G > T |
| W1098X | G > A |
| C524X | C > A |
| G550X | G > T |
| W57X | G > A |
| Q1411X | C > T |
| Q1382X | C > T |
| R1102X | A > T |
| Q685X | C > T |
| W496X | G > A |
| Q715X | C > T |
| L88X | T > A |
| W882X | G > A |
| Q1330X | C > T |
| Q2X | C > T |
| W19X | G > A |
| W216X | G > A |
| Y275X | C > G |
| Q720X | C > T |
| Q30X | C > T |

The split psEONs of the present invention formed from the hybridization of two or more oligonucleotides comprise a single guide region. In some embodiments, the guide region corresponds to the guide region that is located at the 3′ terminal part of a wild-type (wt) H/ACA snoRNA. In one embodiment, the partially duplexed nucleic acid molecule of the split psEON represents the hairpin at the 3′ part of the wt H/ACA snoRNA, wherein the 5′-end of the split psEON starts at any position between the two stems found in the wt H/ACA snoRNA. In some embodiments, partially duplexed nucleic acid molecule of the split psEON comprises one or more additional nucleotides not present in the stem structure of the wt H/ACA snoRNA. In some embodiments, the split psEON of the present invention does not comprise a full H box at its 5′-end, but does comprise a wild type ACA box at its 3′-end. In another preferred aspect, it does comprise a full H box at the 5′-end, but lacks a full length ACA box at the 3′-end. This is in accordance with known interactions of the pseudouridine synthase, dyskerin, two of which bind to the natural H/ACA snoRNAs such that one binds the 5′ hairpin and the H box, while the other binds the 3′ hairpin and the ACA box.

In some embodiments, the split psEONs of the present invention incorporate specific nucleotide modifications to ensure stability as well as proper protein binding and pseudouridylation activity. These changes may vary and may include modifications in the backbone of one or more oligonucleotide molecules that form the split psEON, in the sugar moiety of the nucleotides as well as in the nucleobases. They may also be variably distributed throughout the sequence of one or more oligonucleotide molecules that form the split psEON, depending on the target and on secondary structures. Specific chemical modifications may be needed to support interactions of different amino acid residues within the RNA-binding domains. For example, PS linkages between nucleotides, and/or 2′-OMe modifications may be tolerated in some parts of the split psEON, while in other parts they should be avoided so as not to disrupt crucial interactions of the enzyme with the phosphate and/or 2′—OH groups. The person skilled in the art will be—with the available knowledge from the art and based on the teaching of the present disclosure—capable of determining whether a certain position within the split psEON is suitable for modification and/or whether a certain inter-nucleoside linkage should or should not have a modification. The modifications should also be selected such that they promote stability and/or prevent degradation of the split psEONs. Specific nucleotide modifications may also be necessary to enhance the pseudouridylation activity on substrate RNAs where the target sequence is not optimal for editing.

The present invention, in one aspect, relates to pseudouridylating editing guide RNAs that can be delivered while being embedded in artificial introns that are flanked by exons of a specified and/or particularly selected gene. Through this, the guide RNA can be expressed in mammalian cells from a vector like a plasmid or a viral vector harbouring this exon-intron-exon sequence. It has previously been demonstrated that it is possible to obtain targeted pseudouridylation in a sequence-specific manner using an intron-embedded guide RNA. This approach can now potentially be applied to promote, for instance, PTC suppression as a novel therapy in genetic diseases caused by PTC mutations.

The present invention is exemplified by, but not limited to, reversing the effect of nonsense stop mutations that usually lead to translation termination and mRNA degradation (via Nonsense Mediated Decay, see below). In another aspect, targeted pseudouridylation can act as a means to recode uridine-containing codons as a mean to modulate protein function via amino acid substitution, for instance in crucial protein regions such as protein kinase active centres.

It is known from the art that naturally expressed pseudouridylation guide RNA sequences (box H/ACA snoRNAs) in mammalian cells are often processed from pre-mRNA introns. The assembly process occurs by binding of several proteins with a function in the pseudouridylation into box H/ACA guide RNAs to form snRNPs. This process occurs during transcription and before splicing. After the guide RNA-containing intron is spliced out and de-branched, processing exonucleases degrade the intron at their 5′ and 3′ termini. However, the associated snRNP proteins protect the box H/ACA guide RNA sequences from degradation, allowing the formation of the mature snRNP complex. snoRNA sequences can be inserted in introns of a gene that is not (or may be) its natural environment, such as the human β-globin (Kiss and Filipowicz. 1995) while still leading to fully mature snRNPs. According to the present invention, oligonucleotides for formation of split psEONs can be imbedded in non-host introns flanked by exons of genes. A non-limiting example of a human gene that serves this purpose is the β-globin gene. Such constructs can be administered to and expressed in a mammalian cell, for instance by using a plasmid or viral vector to express oligonucleotides for formation of split psEONs carrying in their pseudouridylation pocket a nucleotide sequence complementary to the target RNA region in a sequence specific way, in a therapeutic setting.

One of the consequences of mutations leading to PTCs in the coding sequence of a gene is the decrease of the mRNA levels. This is due to a mechanism known as the Nonsense-Mediated Decay (NMD), which is a cellular surveillance mechanism in mammals preventing transcripts that were not correctly processed to be translated. It is estimated that one-third of genetic disorders are a result of a mutation leading to a PTC (such as for instance in CF, retinitis pigmentosa (RP), and beta-thalassemia). In a normal scenario, exon-junction complexes (EJCs) are formed during splicing. Then, during the first translation round, ribosomes displace these EJCs. On the other hand, when a PTC is located more than 50-54 nucleotides upstream of the last EJC, the NMD pathway is triggered by formation of a termination complex consisting of EJC-associated NMD factors. When this happens during the first pioneer round of translation and the ribosomes co-exist with at least one EJC downstream their location, this triggers the de-capping and 5′-to-3′ exonuclease activity and also de-adenylation of the tail and 3′-to-5′ exonuclease-mediated transcript decay. In order to tackle the aforementioned genetic disorders, or any disorder that is due to a similar mutation, the inhibition of this pathway in a gene-specific and sequence-specific manner is therefore crucial. The present invention is exemplified by recoding a PTC, which results in an increase of mRNA levels, and in translational read-though of the recoded mRNA into a full-length protein. To assess NMD suppression, a known NMD-inhibition reporter assay (Zhang et al. 1998, RNA 4(7):801-815) can be used, and translational read-through of a gene carrying a PTC can also be assessed.

In some embodiments, when embedded in an intronic sequence, oligonucleotides that hybridize to form a split psEON of the invention can be applied for pseudouridylation in a cell after the exon-intron-exon sequence is administered to the cell. Such may be in the form of a naked nucleic acid. One other way by which such constructs (exon-intron-exon sequences) can be delivered to the cell (either in vitro, ex vivo or in vivo) is by using a delivery vehicle such as a viral vector. One preferred viral vector is based on Adeno-Associated Virus (AAV). Another preferred viral vector is for instance a retroviral vector such as a lentivirus vector and the like. Also, plasmids, artificial chromosomes, and plasmids usable for targeted homologous recombination and integration in the human genome of cells may be suitably applied for delivery of a snoRNA as defined herein.

Typically, when the snoRNA is delivered by a viral vector, it is in the form of an RNA transcript that comprises the sequence of an oligonucleotide according to the invention in a part of the transcript. An AAV vector according to the invention is a recombinant AAV vector and refers to an AAV vector comprising part of an AAV genome comprising an exon-intron-exon sequence according to the invention encapsidated in a protein shell of capsid protein derived from an AAV serotype. Part of an AAV genome may contain the inverted terminal repeats (ITR) derived from an adeno-associated virus serotype, such as AAV1, AAV2, AAV3, AAV4, AAV5, AAV6, AAV7, AAV8, AAV9 and others. Protein shell comprised of capsid protein may be derived from an AAV serotype such as AAV1, 2, 3, 4, 5, 6, 7, 8, 9 and others. A protein shell may also be named a capsid protein shell. AAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV2 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention. Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV 2/5 vector. More preferably, a recombinant AAV vector according to the invention comprises a capsid protein shell of AAV serotype 2 and the AAV genome or ITRs present in said vector are derived from AAV serotype 8; such vector is referred to as an AAV 2/8 vector. More preferably, a recombinant AAV vector according to the invention comprises a capsid protein shell of AAV serotype 2 and the AAV genome or ITRs present in said vector are derived from AAV serotype 9; such vector is referred to as an AAV 2/9 vector. More preferably, a recombinant AAV vector according to the invention comprises a capsid protein shell of AAV serotype 2 and the AAV genome or ITRs present in said vector are derived from AAV serotype 2; such vector is referred to as an AAV 2/2 vector. A nucleic acid molecule harboring an exon-intron-guide RNA-intron-exon sequence according to the invention represented by a nucleic acid sequence of choice is preferably inserted between the AAV genome or ITR sequences as identified above, for example an expression construct comprising an expression regulatory element operably linked to a coding sequence and a 3′ termination sequence. “AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art. The AAV helper functions can be supplied on an AAV helper construct, which may be a plasmid.

Introduction of the helper construct into the host cell can occur e.g. by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the AAV genome present in said AAV vector replication and packaging on the other hand. “AAV helper virus” provides additional functions required for AAV replication and packaging.

Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456. Preferably, an AAV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art. A preferred AAV vector according to the invention is an AAV vector, preferably an AAV2/5, AAV2/8, AAV2/9 or AAV2/2 vector.

### Definitions of Terms as Used Herein

The terms ‘adenine’, ‘guanine’, ‘cytosine’, ‘thymine’, ‘uracil’ and ‘hypoxanthine’ (the nucleobase in inosine) as used herein refer to the nucleobases as such.

The terms ‘adenosine’, ‘guanosine’, ‘cytidine’, ‘thymidine’, ‘uridine’, ‘pseudouridine’ and ‘inosine’, refer to the nucleobases linked to the (deoxy)ribosyl sugar.

The term ‘nucleoside’ refers to the nucleobase linked to the (deoxy)ribosyl sugar.

The term ‘nucleotide’ refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group, well known in the art), a nucleotide including a linker comprising a phosphodiester, phosphotriester, phosphoro(di)thioate, methylphosphonates, phosphoramidate linkers, and the like.

Sometimes the terms adenosine and adenine, guanosine and guanine, cytosine and cytidine, uracil and uridine, thymine and thymidine, inosine and hypo-xanthine, are used interchangeably to refer to the corresponding nucleobase, nucleoside or nucleotide. Pseudouridine is often referred to as ψ, or as 5-ribosyluracil.

Sometimes the terms nucleobase, nucleoside and nucleotide are used interchangeably, unless the context clearly requires differently. The terms ‘ribonucleoside’ and ‘deoxyribonucleoside’, or ‘ribose’ and ‘deoxyribose’ are as used in the art.

Whenever reference is made to an ‘oligonucleotide’, both oligoribonucleotides and deoxyoligoribonucleotides are meant unless the context dictates otherwise. Whenever reference is made to an ‘oligoribonucleotide’ it may comprise the bases A, G, C, U or I. Whenever reference is made to a ‘deoxyoligoribonucleotide’ it may comprise the bases A, G, C, T or I. In a preferred aspect, the EON of the present invention is an oligoribonucleotide that may comprise chemical modifications, and may include deoxynucleotides (DNA) at certain specified positions.

Whenever reference is made to nucleotides in the oligonucleotide, such as cytosine, 5-methylcytosine, 5-hydroxymethylcytosine, Pyrrolocytidine, and β-D-Glucosyl-5-hydroxy-methylcytosine are included; when reference is made to adenine, 2-aminopurine, 2,6-diaminopurine, 3-deazaadenosine, 7-deazaadenosine, 8-azidoadenosine, 8-methyladenosine, 7-aminomethyl-7-deazaguanosine, 7-deazaguanosine, N6-Methyladenine and 7-methyladenine are included; when reference is made to uracil, 5-methoxyuracil, 5-methyluracil, dihydrouracil, pseudouracil, and thienouracil, dihydrouracil, 4-thiouracil and 5-hydroxymethyluracil are included; when reference is made to guanosine, 7-methylguanosine, 8-aza-7-deazaguanosine, thienoguanosine and 1-methylguanosine are included.

Whenever reference is made to nucleosides or nucleotides, ribofuranose derivatives, such as 2′-desoxy, 2′-hydroxy, and 2′-0-substituted variants, such as 2′-O-methyl (2′-OMe), are included, as well as other modifications, including 2′-4′ bridged variants.

Whenever reference is made to oligonucleotides, linkages between two mono-nucleotides may be phosphodiester linkages as well as modifications thereof, including, phosphodiester, phosphotriester, phosphoro(di)thioate, methylphosphonate, phosphor-amidate linkers, and the like.

The term ‘comprising’ encompasses ‘including’ as well as ‘consisting’, e.g. a composition ‘comprising X’ may consist exclusively of X or may include something additional, e.g. X+Y.

The term ‘about’ in relation to a numerical value x is optional and means, e.g. x±10%.

The word ‘substantially’ does not exclude ‘completely’, e.g. a composition which is ‘substantially free from Y’ may be completely free from Y. Where relevant, the word ‘substantially’ may be omitted from the definition of the invention.

The term “complementary” as used herein refers to the fact that the nucleic acid molecule according to the invention hybridizes under physiological conditions to the target RNA sequence and/or to its own internal sequences, especially within the hairpin structure. The term does not mean that each and every nucleotide in the nucleic acid molecule has a perfect pairing with its opposite nucleotide in the target sequence or within the hairpin structure. In other words, while a nucleic acid molecule according to the invention may be complementary to a target sequence, there may be mismatches, wobbles and/or bulges between the nucleic acid molecule of the present invention and the target sequence, while under physiological conditions that nucleic acid molecule still hybridizes to the target sequence such that the cellular enzymes can convert the target uridine to a ψ. The term “substantially complementary” therefore also means that in spite of the presence of the mismatches, wobbles, and/or bulges, the nucleic acid molecule according to the present invention has enough matching nucleotides with the target sequence that under physiological conditions the nucleic acid molecule hybridizes to the target RNA. As shown herein, a nucleic acid molecule may be complementary, but may also comprise one or more mismatches, wobbles and/or bulges with the target sequence, as long as under physiological conditions the nucleic acid molecule of the present invention is able to hybridize to its target.

The term ‘downstream’ in relation to a nucleic acid sequence means further along the sequence in the 3′ direction; the term ‘upstream’ means the converse. Thus in any sequence encoding a polypeptide, the start codon is upstream of the stop codon in the sense strand, but is downstream of the stop codon in the antisense strand.

References to ‘hybridization’ typically refer to specific hybridization, and exclude non-specific hybridization. Specific hybridization can occur under experimental conditions chosen, using techniques well known in the art, to ensure that the majority of stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. Mismatching nucleotides are G-A, C-A, U-C, A-A, G-G, C-C, U-U pairs. In some embodiments nucleic acid molecules according to the present invention comprise fewer than four mismatches, for example 0, 1 or 2 mismatches. Wobble base pairs are: G-U, I-U, I-A, and I-C base pairs.

The term ‘splice mutation’ relates to a mutation in a gene that encodes for a pre-mRNA, wherein the splicing machinery is dysfunctional in the sense that removal of introns from the pre-mRNA is disturbed and due to the aberrant splicing, for instance the translation of a fully functional protein is prevented, either by formation of a dysfunctional protein or by absence of the protein. Often such dysfunctional proteins are degraded rapidly and do not have any functional activity, as discussed herein, and the aberrantly spliced mRNAs may also be rapidly degraded. In a preferred aspect, the splice mutations that are targeted by the nucleic acid molecules of the present invention and through the methods of the present invention are present in the human CFTR gene. The skilled person is aware of methods to determine whether or not normal splicing is restored.

A free (or naked) oligonucleotide according to the present invention may be chemically modified almost in its entirety, for example by providing nucleotides with a 2′-O-methylated sugar moiety (2′-OMe) and/or with a 2′-O-methoxyethyl sugar moiety (2′-MOE).

Various chemistries and modifications are known in the field of oligonucleotides that can be readily used in accordance with the invention. All chemical modifications listed herein may be used in an oligonucleotide that is part of a split psEON as disclosed herein. This includes the use of hydrophobic moieties (such as tocopherol and cholesterol) and cell-specific ligands (such as GalNAc moieties).

The skilled person knows that an oligonucleotide, such as an oligonucleotide that is part of a split psEON as disclosed herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a ‘scaffold modification’. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the oligonucleotide that is part of a split psEON as disclosed herein are disclosed in WO2020/154342, WO2020/154343, and WO2020/154344.

A nucleoside in an oligonucleotide that is part of a split psEON as disclosed herein may be a natural nucleoside (deoxyribonucleoside or ribonucleoside) or a non-natural nucleoside. It is noted that for pseudouridylation of RNA, in which double-stranded RNA is generally the substrate for enzymes, ribonucleosides are considered ‘natural’, while deoxyribonucleosides may then be, for the sake of argument, considered as non-natural, or modified, simply because DNA is not present in the RNA-RNA double stranded (natural) substrate configurations. The skilled person appreciates that when the nucleotide has a natural ribose moiety, it may still be non-naturally modified in the base and/or the linkage.

It is recognized in the art that common limiting factors in oligonucleotide-based therapies are the oligonucleotide's ability to be taken up by the cell (when delivered per se, or ‘naked’ without applying a delivery vehicle such as a viral vector or plasmid), the biodistribution and the resistance to nuclease-mediated breakdown. The skilled person is aware, and it has been described in detail in the art, that a variety of chemical modifications can assist in overcoming such limitations. Examples of such now commonly used chemical modifications are the 2′-OMe, 2′-F and 2′-MOE modifications of the sugar and the use of PS linkages between nucleosides.

The ribose 2′ groups in all nucleotides of the oligonucleotide that is part of a split psEON as disclosed herein can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The 2′-4′ linkage can be selected from many linkers known in the art, such as a methylene linker, amide linker, or constrained ethyl linker (cEt).

An oligonucleotide that is part of a split psEON as disclosed herein may comprise one or more nucleotides carrying a 2′-MOE ribose modification. Also, an oligonucleotide that is part of a split psEON as disclosed herein may comprise one or more nucleotides not carrying a 2′-MOE ribose modification. An oligonucleotide that is part of a split psEON as disclosed herein may comprise a 2′-OMe ribose modification at a position that does not comprise a 2′-MOE ribose modification. An oligonucleotide that is part of a split psEON as disclosed herein may comprise deoxynucleotides at positions that do not comprise a 2′-MOE or a 2′-OMe ribose modification, or other 2′ ribose substitution. An oligonucleotide that is part of a split psEON as disclosed herein may comprise one or more nucleotides comprising a 2′ substitution comprising a 2′-MOE, 2′-OMe, 2′-OH, 2′-deoxy, TNA, 2′-fluoro (2′-F), 2′,2′-difluoro (diF) modification, 2′-fluoro-2′-C-methyl modification, or a 2′-4′-linkage (i.e., a bridged nucleic acid such as a locked nucleic acid (LNA). Other nucleic acid monomers that may be used in an oligonucleotide that is part of a split psEON as disclosed herein are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications that may present in an oligonucleotide that is part of a split psEON as disclosed herein are known in the art, including but not limited to the modifications outlined in detail in WO2016/097212, WO2017/220751, WO2018/041973, WO2018/134301, WO2019/219581, WO2019/158475, and WO2022/099159. In all cases, the modifications should be compatible with pseudouridylation such that the split psEON fulfils its role as a complex that can form a double stranded complex with the target RNA and mediate pseudouridylation. Where a monomer in an oligonucleotide that is part of a split psEON as disclosed herein comprises an unlocked nucleic acid (UNA) ribose modification, that monomer can have a 2′ position comprising the same modifications discussed above, such as a 2′-MOE, a 2′-OMe, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a locked nucleic acid (LNA)). In an aspect, an oligonucleotide that is part of a split psEON as disclosed herein comprises at least one nucleotide comprising a threose nucleic acid (TNA) ribose modification. In one aspect, an oligonucleotide that is part of a split psEON as disclosed herein comprises at least one nucleotide with a sugar moiety that comprises a 2′-fluoro (2′-F) modification.

A base, sometimes called a nucleobase, is generally adenine, cytosine, guanine, thymine or uracil, or a derivative thereof. A nucleobase is defined as a moiety that can bond to another nucleobase through H-bonds, polarized bonds or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar. The nucleobases in an oligonucleotide that is part of a split psEON as disclosed herein can be adenine, cytosine, guanine, thymine, or uracil or any other moiety able to interact with another nucleobase through H-bonds, polarized bonds (such as CF) or aromatic electronic interactions. The nucleobases at any position in an oligonucleotide that is part of a split psEON as disclosed herein can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application, and may be worked out in accordance with the wishes and preferences of those of skill in the art.

A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-OMe, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-O-(haloalkyl)methyl such as 2′-O-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl](MOCE), 2′-O-[2-N-methylcarbamoyl)ethyl](MCE), 2′-O-[2-(N,N-dimethylcarbamoyl)ethyl](DCME); 2′-halo e.g. 2′-F, FANA; 2′-O-[2-(methylamino)-2-oxoethyl](NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-0,4′-C) constrained ethyl (cEt) BNA monomer, a (2′-0,4′-C) constrained methoxyethyl (cMOE) BNA monomer, a 2′,4′-BNANC(NH) monomer, a 2′,4′-BNANC(NMe) monomer, a 2′,4′-BNANC(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-I-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

A nucleoside is generally connected to neighboring nucleosides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the ‘backbone’ of the oligonucleotide. Because phosphodiester bonds connect neighboring monomers together, they are often referred to as ‘backbone linkages’. It is understood that when a phosphate group is modified so that it is instead an analogous moiety such as a phosphorothioate, such a moiety is still referred to as the backbone linkage of the monomer. This is referred to as a ‘backbone linkage modification’. In general terms, the backbone of an oligonucleotide comprises alternating scaffolds and backbone linkages.

As outlined in detail herein, a naked oligonucleotide that is part of a split psEON as disclosed herein comprises at least one, preferably multiple linkage modifications. It is generally more preferred that an oligonucleotide that is part of a split psEON as disclosed herein comprises linkage modifications at most, and potentially all positions if the oligonucleotide is capable of mediating pseudouridylation. A linkage modification can be, but is not limited to, a modified version of the phosphodiester present in RNA, such as phosphorothioate (PS), chirally pure PS, (R)-PS, (S)-PS, methyl phosphonate (MP or MeP), chirally pure MP, (R)-MP, (S)-MP, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, PS prodrug, alkylated PS, H-phosphonate, ethyl phosphate, ethyl PS, boranophosphate, borano PS, methyl boranophosphate, methyl borano PS, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts, deprotonated, protonated, tautomeric, and free acid forms are also included, as well as 3′→3′ and 2′→5′ linkages. An AON as disclosed herein may also comprise one or more linkage modifications according to the structure of formula (I)

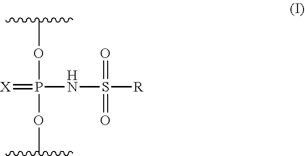

wherein:
X═O or S; and
R=an aryl, a substituted aryl, a heterocycle, a substituted heterocycle, an aromatic heterocycle, a substituted aromatic heterocycle, a C1-C6 alkoxy, a substituted C1-C6 alkoxy, a C1-C20 alkyl, a substituted C1-C20 alkyl, a C1-C6 alkenyl, a C1-C6 substituted alkenyl, a C1-C6 alkynyl, a substituted C1-C6 alkynyl, or a conjugate group. In a preferred embodiment, X═O and R=methyl and the linkage modification is referred to as mesyl phosphoramidate, MsPA or PNms.

In a preferred aspect, an oligonucleotide that is part of a split psEON as disclosed herein comprises an internucleoside linkage of the structure of formula (I), wherein X═O and R═CH3, which linkage is generally referred to herein as a PNms linkage (mesyl phosphoramidate). In other preferred aspects, R equals one of the following structures (a), (b), (c), (d), (e), (f), (g), (h), or (i):

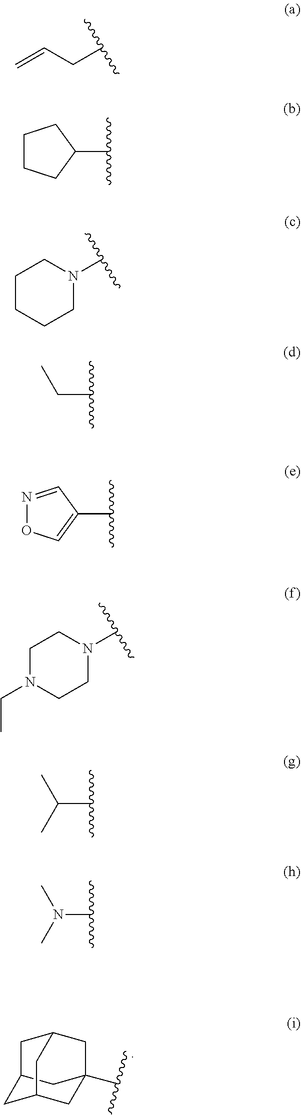

An oligonucleotide that is part of a split psEON as disclosed herein may comprise a substitution of one of the non-bridging oxygens in the phosphodiester linkage. This modification slightly destabilizes base pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS. Particularly preferred are internucleoside linkages that are modified to contain a PNms. Particularly preferred are internucleoside linkages that are modified to contain a PNdmi. The regular internucleosidic linkages between the nucleotides may be altered by mono- or di-thioation of the phosphodiester bonds to yield PS esters or phosphorodithioate esters, respectively. Other modifications of the internucleosidic linkages are possible, including amidation and peptide linkers. The skilled person can determine for what target RNA nucleic acid molecule the oligonucleotide comprises a certain linkage modification at each linkage position of an oligonucleotide that is part of a split psEON as disclosed herein to generate the most effective and most stable split psEON compound.

Many of the non-naturally occurring modification of the linkage, such as PS, are chiral. This means that there are Rp and Sp configurations, known to the person skilled in the art. In one embodiment, the chirality of the PS linkages is controlled, which means that each of the linkages is either in the Rp or in the Sp configuration, whichever is preferred. The choice of an Rp or Sp configuration at a specified linkage position may depend on the target sequence and the efficiency of binding and induction of causing pseudouridylation of the target U. However, if such is not specifically desired, a composition may comprise split psEONs as active compounds with both Rp and Sp configurations at a certain specified linkage position. Mixtures of such split psEONs are also feasible, wherein certain positions have preferably either one of the configurations, while for other positions such does not matter. In one aspect, an oligonucleotide that is part of a split psEON as disclosed herein comprises one or more (chirally pure or chirally mixed) PS linkages. In one aspect, an oligonucleotide that is part of a split psEON as disclosed herein comprises one of more (chirally pure or chirally mixed) phosphoramidate (PN) linkages. In one aspect, an oligonucleotide that is part of a split psEON as disclosed herein comprises one or more (chirally pure or chirally mixed) PNms linkages. In one aspect, a PN linkage connects the terminal two nucleotides on each end of the oligonucelotide. An oligonucleotide that is part of a split psEON as disclosed herein may also comprise linkage modifications at all positions that are not chirally controlled. An oligonucleotide that is part of a split psEON as disclosed herein may also comprise one or more naturally occurring internucleoside linkages. The choice and number of modified linkages may depend on the specific target, the sequence, the length, and the stability of the oligonucleotide observed in a particular cell type of interest, which can be assessed by methods known to the person skilled in the art. In one aspect, at least one, at least two, at least three, or at least four internucleoside linkages between the 5′ and/or the 3′ terminal two, three, four, or five nucleosides respectively of an oligonucleotide that is part of a split psEON as disclosed herein are modified internucleoside linkages. In one aspect, an oligonucleotide that is part of a split psEON as disclosed herein comprises at least one MP internucleoside linkage according to the structure of formula (II):

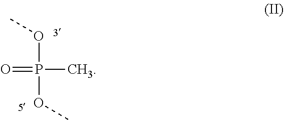

In one aspect, an oligonucleotide that is part of a split psEON as disclosed herein comprises at least one PNdmi linkage. A PNdmi linkage as preferably used in an oligonucleotide that is part of a split psEON as disclosed herein has the structure of formula (III)

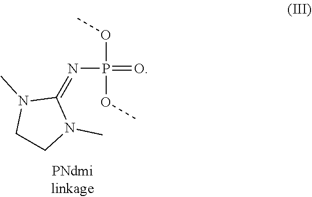

Other internucleoside linkages that may be used in an oligonucleotide that is part of a split psEON as disclosed herein are those that are disclosed in WO2023/278589.

In one aspect, an oligonucleotide that is part of a split psEON as disclosed herein comprises at least one phosphonoacetate and/or at least one phosphonoacetamide internucleoside linkage.

The internucleosidic linkages may be replaced in full or in part by peptidic linkages to yield in peptidonucleic acid sequences and the like. Alternatively, or in addition, the nucleobases may be modified by (de)amination, to yield inosine or 2′6′-diaminopurines and the like. A further modification may be methylation of the C5 in the cytidine moiety of the nucleotide, to reduce potential immunogenic properties known to be associated with CpG sequences.

The degree of recruiting and redirecting the pseudouridylation entities resident in the cell may be regulated by the dosing and the dosing regimen of the snoRNA. This is something to be determined by the experimenter (in vitro) or the clinician, usually in phase I and/or II clinical trials.

The invention concerns the modification of target RNA sequences in eukaryotic, preferably metazoan, more preferably mammalian cells. In principle the invention can be used with cells from any mammalian species, but it is preferably used with a human cell. The invention can be used with cells from any organ e.g. skin, lung, heart, kidney, liver, pancreas, gut, muscle, gland, eye, brain, blood and the like. The invention is particularly suitable for modifying sequences in cells, tissues or organs implicated in a diseased state of a (human) subject, for instance when the human subject suffers from CF. Such cells include but are not limited to epithelial cells of the lung. The cell can be located in vitro or in vivo. One advantage of the invention is that it can be used with cells in situ in a living organism, but it can also be used with cells in culture. In some embodiments cells are treated ex vivo and are then introduced into a living organism (e.g. re-introduced into an organism from whom they were originally derived). The invention can also be used to edit target RNA sequences in cells within a so-called organoid. Organoids can be thought of as three-dimensional in vitro-derived tissues but are driven using specific conditions to generate individual, isolated tissues (e.g. see Lancaster and Knoblich. 2014, Science 345 (6194):1247125). In a therapeutic setting they are useful because they can be derived in vitro from a patient's cells, and the organoids can then be re-introduced to the patient as autologous material which is less likely to be rejected than a normal transplant. The cell to be treated will generally have a genetic mutation. The mutation may be heterozygous or homozygous. The invention will typically be used to modify point mutations.

The invention is used to make a change in a target RNA sequence in a eukaryotic cell through the use of an oligonucleotide that is capable of targeting a site to be edited and recruiting RNA editing entities resident in the cell to bring about the editing reaction(s). The target RNA sequence may comprise a mutation that one may wish to correct or alter, such as a point mutation (a transition or a transversion). The target RNA may be any cellular or viral RNA sequence, but is more usually a pre-mRNA or an mRNA with a protein coding function. The target sequence is endogenous to the eukaryotic, preferably mammalian, more preferably human cell.

The amount of nucleic acid to be administered, the dosage and the dosing regimen can vary from cell type to cell type, the disease to be treated, the target population, the mode of administration (e.g. systemic versus local), the severity of disease and the acceptable level of side activity, but these can and should be assessed by trial and error during in vitro research, in pre-clinical and clinical trials. The trials are particularly straightforward when the modified sequence leads to an easily-detected phenotypic change. It is possible that higher doses of nucleic acid could compete for binding to a nucleic acid editing entity within a cell, thereby depleting the amount of the entity which is free to take part in pseudouridylation, but routine dosing trials will reveal any such effects for a given nucleic acid molecule and a given target.

One suitable trial technique involves delivering the nucleic acid molecule according to the invention to cell extracts, cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained and future delivery can be performed without needing to take biopsy samples. A method of the invention can thus include a step of identifying the presence of the desired change in the cell's target RNA sequence, thereby verifying that the target RNA sequence has been modified. The change may be assessed on the level of the protein (length, glycosylation, function or the like), or by some functional read-out, such as a(n) (inducible) current, when the protein encoded by the target RNA sequence is an ion channel, for example. In the case of CFTR function, an Ussing chamber assay or an NPD test in a mammal, including humans, are well known to a person skilled in the art to assess restoration or gain of function.

After pseudouridylation has occurred in a cell, the modified RNA can become diluted over time, for example due to cell division, limited half-life of the edited RNAs, etc. Thus, in practical therapeutic terms a method of the invention may involve repeated delivery of an oligonucleotide until enough target RNAs have been modified to provide a tangible benefit to the patient and/or to maintain the benefits over time.

Nucleic acid sequences (oligonucleotides, split psEONs, vectors) of the invention are particularly suitable for therapeutic use, and so the invention provides a pharmaceutical composition comprising an oligonucleotide or carrier vector of the invention and a pharmaceutically acceptable carrier or solvent. In some embodiments of the invention the pharmaceutically acceptable carrier or solvent can simply be a saline solution. This can usefully be isotonic or hypotonic, particularly for pulmonary delivery. The invention also provides a delivery device (e.g. syringe, inhaler, nebuliser) which includes a pharmaceutical composition of the invention.

The invention also provides an oligonucleotide of the invention for use in a method for making a change in a target RNA sequence in a mammalian, preferably a human cell, as described herein. Similarly, the invention provides the use of a nucleic acid molecule, such as an oligonucleotide or expression construct or vector of the invention in the manufacture of a medicament for making a change in a target RNA sequence in a mammalian, preferably a human cell, as described herein.

The invention also relates to a method for the pseudouridylation of at least one specific target uridine present in a target RNA sequence in a cell, the method comprising the steps of: providing the cell with a combination of nucleic acid molecules or a split psEON according to the invention; allowing uptake by the cell of the nucleic acid molecules (or the split psEON); allowing annealing of the nucleic acid molecules (or the split psEON) to the target RNA sequence; allowing the formation of a pseudouridylation-competent RNP with the introduced guide RNA incorporated, to pseudouridylate the target uridine in the target RNA sequence to ψ; and optionally identifying the presence of the ψ in the RNA sequence. The oligonucleotide molecules may be manufactured and delivered as such, but may also be, as disclosed herein, administered as a partially duplexed nucleic acid molecule (i.e. as a split psEON.)

Introduction of the nucleic acid molecule according to the present invention into the cell is performed by general methods known to the person skilled in the art. After pseudouridylation, the read-out of the effect (alteration of the target RNA sequence) can be monitored through different ways. Hence, the identification step of whether the desired pseudouridylation of the target uridine has indeed taken place depends generally on the position of the target uridine in the target RNA sequence, and the effect that is incurred by the presence of the uridine (point mutation, PTC). Hence, in a preferred aspect, depending on the ultimate effect of U to ψ conversion, the identification step comprises: assessing the presence of a functional, elongated, full length and/or wild type protein; assessing whether splicing of the pre-mRNA was altered by the pseudouridylation; or using a functional read-out, wherein the target RNA after the pseudouridylation encodes a functional, full length, elongated and/or wild type protein. The functional assessment for each of the diseases mentioned herein will generally be according to methods known to the skilled person.

The nucleic acid molecule, combination thereof (e.g., split psEON) or vector according to the invention is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g. through nebulization), intranasally, orally, by injection or infusion, intravenously, subcutaneously, intra-dermally, intra-cranially, intravitreally, intramuscularly, intra-tracheally, intra-peritoneally, intra-rectally, and the like. Administration may be in solid form, in the form of a powder, a pill, or in any other form compatible with pharmaceutical use in humans. The invention is particularly suitable for treating genetic diseases, such as CF.

In some embodiments the nucleic acid molecule, combination thereof (e.g., split psEON) or vector according to the invention can be delivered systemically, but it is more typical to deliver an oligonucleotide to cells in which the target sequence's phenotype is seen. For instance, mutations in CFTR cause CF which is primarily seen in lung epithelial tissue, so with a CFTR target sequence it is preferred to deliver the oligonucleotide construct specifically and directly to the lungs. This can be conveniently achieved by inhalation e.g. of a powder or aerosol, typically via the use of a nebuliser. Especially preferred are nebulizers that use a so-called vibrating mesh, including the PARI eFlow (Rapid) or the i-neb from Respironics. It is to be expected that inhaled delivery of oligonucleotide constructs according to the invention can also target these cells efficiently, which in the case of CFTR gene targeting could lead to amelioration of gastrointestinal symptoms also associated with CF. In some diseases the mucus layer shows an increased thickness, leading to a decreased absorption of medicines via the lung. One such a disease is chronical bronchitis, another example is CF. A variety of mucus normalizers are available, such as DNases, hypertonic saline or mannitol, which is commercially available under the name of Bronchitol. When mucus normalizers are used in combination with pseudouridylating oligonucleotide constructs, such as the split psEON constructs according to the invention, they might increase the effectiveness of those medicines. Accordingly, administration of an oligonucleotide construct according to the invention to a subject, preferably a human subject is preferably combined with mucus normalizers, preferably those mucus normalizers described herein. In addition, administration of the oligonucleotide constructs according to the invention can be combined with administration of small molecule for treatment of CF, such as potentiator compounds for example Kalydeco (ivacaftor; VX-770), or corrector compounds, for example VX-809 (lumacaftor) and/or VX-661. Alternatively, or in combination with the mucus normalizers, delivery in mucus penetrating particles or nanoparticles can be applied for efficient delivery of pseudouridylating molecules to epithelial cells of for example lung and intestine. Accordingly, administration of an oligonucleotide construct according to the invention to a subject, preferably a human subject, preferably uses delivery in mucus penetrating particles or nanoparticles. Chronic and acute lung infections are often present in patients with diseases such as cystic fibrosis. Antibiotic treatments reduce bacterial infections and the symptoms of those such as mucus thickening and/or biofilm formation. The use of antibiotics in combination with oligonucleotide constructs according to the invention could increase effectiveness of the pseudouridylation due to easier access of the target cells for the oligonucleotide construct. Accordingly, administration of an oligonucleotide construct according to the invention to a subject, preferably a human subject, is preferably combined with antibiotic treatment to reduce bacterial infections and the symptoms of those such as mucus thickening and/or biofilm formation. The antibiotics can be administered systemically or locally or both. For application in CF patients the oligonucleotide constructs according to the invention, or packaged or complexed oligonucleotide constructs according to the invention may be combined with any mucus normalizer such as a DNase, mannitol, hypertonic saline and/or antibiotics and/or a small molecule for treatment of CF, such as potentiator compounds for example ivacaftor, or corrector compounds, for example lumacaftor and/or VX-661. To increase access to the target cells, Broncheo-Alveolar Lavage (BAL) could be applied to clean the lungs before administration of the oligonucleotide according to the invention.

### EXAMPLES

### Example 1: Design of Pseudouridylating Guide Oligonucleotides Derived from the Structure of Small Nucleolar RNA (snoRNA) for the Conversion of a Specific Uridine in ACA19 Target RNA to ψ

The inventors of the present invention questioned whether it would be possible to induce pseudouridylation using a shortened box H/ACA snoRNA. For this, a pseudouridylation guide RNA was designed in which the 5′ hairpin of the full-length ACA19 snoRNA, and most of the H box were removed (FIG. 4). The shortened guide RNA and the full-length snoRNA were produced by in vitro transcription using T7 RNA polymerase. For testing in cell lysates, a short substrate RNA for these guides was produced either by in vitro transcription using T7 RNA polymerase (in the presence of [α-³²P]UTP) or by two-piece ligation. For the latter, first, a synthetic RNA oligonucleotide, terminating at its 5′ end in the uridine to be pseudouridylated, was radioactively labelled at the 5′ hydroxyl group with a [γ-³²P]phosphate using T4 polynucleotide kinase. The radioactively labelled RNA oligonucleotide was then ligated at its 5′ end to the 3′ hydroxyl group of another RNA oligonucleotide to form the substrate RNA. This was done by annealing the RNA oligonucleotides with a 30-nt bridging DNA oligonucleotide, such that the 5′ half (15 nt) of the bridging DNA oligo base-paired with the 3′ RNA fragment and the 3′ half (15-nt) of the bridging DNA oligo base-paired with the 5′ RNA fragment, and then providing them with T4 DNA ligase to covalently link the RNA oligonucleotides. The ligated, radioactively labelled RNA substrate then has the sequence of 5′-AGGGGAACCCCACAGUCGAACCAAAACAAA-3′ (SEQ ID NO:1), in which the target uridine (containing the radioactive phosphate on its 5′ side) is underlined. This substrate RNA was then purified from the other nucleic acids by separating it, cutting it from a denaturing polyacrylamide gel, eluting it from the gel and finally concentrating it by ethanol precipitation.

For preparing the cell lysate for testing the pseudouridylation, HeLa cells were grown on standard 10-cm cell culture dishes in DMEM with 10% FBS to a confluency of 80-100%, after which they were collected by scraping, and washed with phosphate buffered saline. Thereafter, 200 μl extraction buffer (25% glycerol, 0.42 M NaCl, 1.5 mM MgCl₂, 0.2 mM EDTA, 20 mM HEPES (pH 7.9), 0.5 mM DTT and 0.5 mM Phenylmethane-sulfonyl Fluoride) was added on the cells, which were then vortexed in the presence of sterile glass beads for 30 sec three times, each time followed by a 30 sec incubation on ice. The cell debris was removed by centrifugation, and the supernatant was collected for use in the pseudouridylation assay.

For pseudouridylation assays, incubation buffer containing 200 mM Tris-HCl, pH 8.0, 200 mM ammonium acetate, 10 mM MgCl₂,4 mM DTT, and 0.2 mM EDTA, as supplemented with 200 ng of the full-length H/ACA snoRNA or the shortened guide RNA, substrate RNA according to its relative radioactivity (5000 counts per minute), 250 ng yeast tRNA, and HeLa cell extract (final concentration 20%) were mixed and incubated at 37° C. for 40 min. RNA was then isolated from the reactions by phenol-chloroform extraction and ethanol precipitation. The RNA was then incubated with P1 nuclease (~300 ng) in 20 mM sodium acetate buffer, pH 5.2 for 1 h at 37° C. to degrade the RNA and release the individual nucleotides. These were then separated by thin-layer chromatography (with the solvent volume ratios 70:15:15 for isopropyl alcohol:HCl:water respectively), and the radioactive nucleotides were then imaged by autoradiography. Due to the different migration of the labelled uridines and pseudouridines derived, the conversion of the labelled substrate uridine into pseudouridine can be clearly observed with both the full-length H/ACA snoRNA and the shortened guide RNA. FIG. 5 shows that when a shortened pseudouridylating guide oligonucleotide is used, the efficiency of pseudouridylation is at least comparable to what is seen with the full length versions of the guide RNAs.

Then, it was investigated whether chemical modifications made in the shortened ACA19 guide RNA would be compatible with the target RNA engagement, and the formation of a catalytically active pseudouridylation complex. The positions of the chemical modifications were selected as described herein. The substrate RNA and the experimental assays used were the same as for the above comparison of the full-length H/ACA snoRNA or the shortened guide RNA. FIG. 6 shows the four ACA19 pseudouridylating editing oligonucleotides (psEONs) and the positions of the chemical modifications that were introduced: black dots indicate 2′-OMe modifications on the ribose moiety of that nucleotide, and open dots indicate PS modification of the linkage between the two riboses. FIG. 7 shows the result of pseudouridylation using the fourACA19 psEONs in parallel with the corresponding guide RNA (lacking chemical modifications) as a positive control and no guide RNA as a negative control. U and ψ indicate the migration of uridine and pseudouridine in the thin layer chromatography, respectively. Clearly, the chemically modified psEONs were able to support conversion of uridine to pseudouridine, and the first psEON even gives a much stronger pseudouridylation signal in comparison to the positive control. The enhanced effect could be due to specific interactions of the chemical modifications of the psEON within the enzymatically active complex, or due to other effects, such as increased resistance to nucleases in the cell lysates, each of which can contribute to the pseudouridylation efficiency and/or rate.

### Example 2: Design of psEONs for the Conversion of a Specific Uridine in a Premature Termination Codon in Human CFTR

It was investigated whether a uridine in a premature termination codon (PTC) in the human CFTR gene could be converted to tp. As an example the CFTR-G542X mutation was selected. Procedures to test this were as described in example 1.

The substrate RNA was constructed similarly as in example 1 (two-piece ligation), with the final target sequence being 5′-GACAAUAUAGUUCUUUGAGAAGGUGGAAUC-3′ (labelled target uridine underlined; SEQ ID NO:2).

FIG. 8 shows the four CFTR-G542X psEONs and the positions of the chemical modifications that were introduced: black dots indicate 2′-OMe modifications on the ribose moiety of that nucleotide, and open dots indicate PS modification of the linkage between the two riboses. FIG. 9 shows the result of pseudouridylation using the four CFTR-G542X psEONs in parallel with the corresponding guide RNA (lacking chemical modifications) as a positive control and no guide RNA as a negative control. U and 4) indicate the migration of uridine and pseudouridine in the thin layer chromatography, respectively. The results clearly indicate that three out of four psEONs outperform the positive control, once again showing that chemical modifications at the indicated positions increase the rate and/or efficiency of pseudouridylation in mammalian cell lysates.

### Example 3: Design of snoRNAs for the Conversion of a Specific Uridine in a Premature Termination Codon in Mouse Idua RNA

It was investigated whether a uridine in a PTC in the mouse Idua RNA could be converted to ψ. As an example the Idua-W392X mutation in the mouse RNA was selected, which corresponds to the human IDUA W402X mutation known to cause Hurler syndrome. For this, the substrate RNA was constructed similarly as in Example 1 (two-piece ligation), with the final sequence of 5′-GAUGGAGAACAACUCUAGGCAGAGGUCUCA-3′ (labelled target uridine underlined; SEQ ID NO:3).

FIG. 10 shows the four Idua-W392X psEONs and the positions of the chemical modifications that were introduced: black dots indicate 2′-OMe modifications on the ribose moiety of that nucleotide, and open dots indicate PS modification of the linkage between the two riboses. FIG. 11 shows the result of pseudouridylation using the four Idua-W392X psEONs in parallel with the corresponding guide RNA (lacking chemical modifications) as a positive control and no guide RNA as a negative control. U and ψ indicate the migration of uridine and pseudouridine in the thin layer chromatography, respectively. Here, the intensity of the negative control is very low due to loss of the sample during the precipitation step after the pseudouridylation reaction. Nonetheless, the results clearly indicate that all of the psEONs are capable of supporting the conversion of a uridine to pseudouridine, and even outperform the positive control, once again showing that chemical modifications at the indicated positions increase the rate and/or efficiency of pseudouridylation in mammalian cell lysates.

### Example 4: Targeted Pseudouridylation of a Premature Termination Codon-Containing Vector Using Intronic-Embedded Guide RNAs

Cloning of the Exon1/Intron-guide RNA-Intron/Exon2 constructs “puglntron-IDUA” and “pugIntronOpt-IDUA”

The puglntron-IDUA plasmid construct was based on the original backbone pdRLuc-GI (Woeller et al. 2008. EMBO Reports 9(5):446-451) and was generated by PCR amplification and site-directed mutagenesis, using Agilent's PfuUltra II fusion HS DNA polymerase. The puglntron plasmid was generated by first inserting restriction sites SalI and PstI into the first intron of human β-globin in the parental pdRLuc-GI vector. The following primers were used: SDM1-GLintron1-Sal-Pst: 5′-GTAAGTCGACGAATTCTGCAGGCTGCTGGTGG-3′ (SEQ ID NO:13) and SDM2-GLintron1-Sal-Pst: 5′-GCCTGCAGAATTCGTCGACTTACCTGCCCAGG-3′ (SEQ ID NO:14). Then, the guide RNA was generated by PCR using the following SalI/PstI containing primers (an extra Py-rich sequence for splicing was also included): pug-intron-FwdSalI: 5′-GTTGTCGAC GTGGGAGATTCT-3′ (SEQ ID NO:15) and pug-intron-RevExPstI: 5′-AATCTGCAGG GGAAAAGAGAGAGTCAACCTGTCTGCCTCGT-3′ (SEQ ID NO:16). First, using a HindIII site located downstream the PstI restriction site, but still in the intronic region, the SalI-HindIII fragment of the parental vector was pasted into an intermediate vector (pEGFP-C3). The guide RNA PCR product digested with SalI-PstI was then inserted into this intermediate vector. Finally, the SalI-HindIII fragment was cloned back into the puglntron expression parental vector. FIG. 12A shows the construct with the upstream CMV promoter, exon 1 (E1) and exon 2 (E2) and the puglntron-IDUA insert in the intronic part, with the SalI and PstI sites indicated. FIG. 13 shows the sequence of the entire puglntron-IDUA plasmid (SEQ ID NO: 11). A further optimized version of this construct (creating a more suitable 3′ splice site sequence) was made by running a PCR using the pug-intron-FwdSalI as the forward primer (see above) and the following primer as the reverse primer (the additional two nucleotides in comparison to the pug-intron-RevExPstI reverse primer, see above, creating an additional 5′-AC-3′ sequence at the 3′ splice site, are underlined): pug-intronOPT-RevExPstI: 5′-AATCTGCAGGGGAAAAGAGAGAGTCAGTACCTGTCTGCCTC-3′ (SEQ ID NO:21). The cloning steps as indicated above were repeated to generate the optimized plasmid pugIntronOpt-IDUA, for which the full sequence is given in SEQ ID NO:22 (comprising the additional 5′-AC-3′ directly downstream of the intron, in comparison to SEQ ID NO:11). The negative control pugCFTR was the original version of the guide RNA which was used for the PCR template of the puglntron. This construct was made from three overlapping DNA oligos, of which backbone is originated from pugU2-34/44, for CFTR: pug-NBD1-F1HindIII 5′-ATTAAGCTTGT GTGGGAGATTCTTCTTCGGACAGAGAGAAACTCTGCTGTG-3′ (SEQ ID NO:27), pug-NBD1-R1 5′-CTGCTGTGTCTGAAAGAAGATCTCCCTATAGTGACCCTGCCTTACCTTCTCCGGGAC GAA-3′ (SEQ ID NO:28) and pug-NBD1-R2BamHI 5′-ATGGATCCACCTGTCTGCC TCGTATTCTTCCGTTACGATTTCTCTCATTTCGTCCCGG-3′ (SEQ ID NO:29), and for IDUA: pug-mmidua-F1HindIII 5′-ATTAAGCTTGTGTGGGAGATTCTGCCTCGGACAGAGAGAAACTCT GCTGTG-3′ (SEQ ID NO:30), pug-mmidua-R1 5′-TTCGTCCCGGGGCAGAGAAGGCAGG GTCACTATAGGGAGATCAACTCTCAGACACAGCAG-3′ (SEQ ID NO:31) and pug-mmidua-R2BamHI 5′-ATGGATCCACCTGTCTGCCTCgtaAACTCCCGTTACGATTTCTCTCATTTC GTCCCGG-3′ (SEQ ID NO:32). Then, the 3 piece-PCR products were digested with HindIII and BamHI and cloned into pcDNA3.1/Zeo(+).

Cloning of the Target Plasmid “GL-IDUA Swap” Comprising the Human β-Globin Gene Harbouring a mmIDUA-W392X PTC Mutation:

As target sequence for pseudouridylation the construct expressing the human β-globin gene was used (WT and TER; Woeller et al. 2008). In the TER version of the plasmid, which contains a PTC in globin codon 39, the target sequence (being the substrate for the guide RNA) was swapped by the CFTR-G542X mutation (serving as a negative control target plasmid) and the mmIDUA-W392X mutation (target for the puglntron-IDUA plasmid) by site-directed mutagenesis using the PfuUltra II fusion HS DNA polymerase according to the manufacturer's protocols (Agilent technologies). In order to swap the 33 nt nonsense region (position −15 to PTC (3 nt) to position+15) in the original pFLAG2CMV2-HBB construct with the 33 nt mmIDUA-W392X target sequence (marked in bold in FIG. 14), the following primers were used for amplification: SDM-GL39-SWAPto-mmidua-1: 5′-TGGTGGATGGAGAACAACTCTAGGCAGAGGTCTCAAAGTT TGGGGATCTGTCCACTCC-3′ (SEQ ID NO:17) and SDM-GL39-SWAPto-mmidua-2: 5′-CCAAACTTTGAGACCTCTGCCTAGAGTTGTT CTCCATCCACCAGCAGCCTAAGGGTGG-3′ (SEQ ID NO:18). For the generation of the CFTR-G542X mutation containing swapped plasmid (GL-CFTR swap) the following primers were used: SDM-GL39-SWAPto-NBD1-1 5′-TGGTGGACAATATAGTTCTTTGAGAAGGTGGAATCACATTTGGGGATCTGTCCACTCC-3′ (SEQ ID NO:33) and SDM-GL39-SWAPto-NBD1-2 5′-CCAAATGTGATTCCACCTTCTC AAAGAACTATATTGTCCACCAGCAGCCTAAGGGTGG-3′ (SEQ ID NO:34).

### Cell Culture and Transfection Protocol:

HEK293T and HeLa cells were cultured in DMEM+10% FBS. Transient transfections were performed using polyethylenimine HCl PEI MAX 40000 (PolySciences) as a stock solution of 1 mg/mL (pH7). Cells were grown in 6-well dishes up a high confluency (90-100%). To prepare the transfection solution, 150 μL of Opti-MEM (serum-free) was mixed with 9 μL of PEI stock solution and the resulting mixture was incubated at RT for 5 min. Following this, 100 ng substrate plasmid DNA (wild-type or PTC-containing) and 2 μg guide RNA-expressing plasmid DNA was added to the aforementioned PEI/medium mix. The resulting solution was incubated at RT for 15 min after which the mixture was added directly to each well.

### RT-PCR:

HEK293T cells were transfected with GL39-IDUA swap substrate plasmid with or without pugIntronOpt-IDUA guide expressing plasmid as given above. Total RNA was isolated using the TRIzol™ Reagent (Invitrogen). Reverse transcription was carried out with AMV Reverse Transcriptase (Promega) and the RT product was then amplified by PCR with GoTaq® Green Master Mix (Promega), using 15 to 23 cycles. The target mRNAs for pseudouridylation were detected using RT-PCR with the following primer pair: Forward primer: RLuc-GI ex1 S4: 5′-TCTGCCGTTACTGCCCTGTG-3′ (SEQ ID NO:19) and Reverse primer: PE-mmiduaPTC+16: 5′-CTTTGAGACCTCTGCC-3′ (SEQ ID NO:20). 5S rRNA was detected for normalization purposes with the following primer pair: 5SFwd: 5′-GCCATACCACCCTGAACG-3′ (SEQ ID NO:23) and 5SRev: 5′-AGCTTCCGAGAT CAGACGAG-3′ (SEQ ID NO:24). RT-PCR products were separated by gel electrophoresis and quantified by Image Studio Lite (LI-COR). Results are given in FIG. 15 and show that when the pugIntronOpt-IDUA plasmid (here pugIntOptIDUA) was transfected with the target plasmid GL39-IDUA swap (here GL391DUA) in HEK293T cells, a RT-PCR product using the primers given above could be detected (given by arrow GL39) after 18 cycles but not yet after 15 cycles. Almost no product could be detected when the GL39-IDUA swap plasmid was transfected without intron-carried guide RNA expressing plasmid. This indicates that the amount of mRNA from the GL39-IDUA swap plasmid was higher in these cells than when no guide RNA was introduced. Indeed, guide-induced RNA pseudouridylation suppressed NMD and upregulated the intact mRNA level 37-fold (0.00437 in lane 2; 0.1617 in lane 4, normalized by 5S control).It is therefore concluded that the guide RNA (from the pugIntronOpt-IDUA plasmid) is capable of giving read-through, thereby suppressing NMD, and that pseudouridylation has occurred. Similar results were obtained after 20 to 23 PCR cycles were performed, and when using a plasmid carrying a CFTR-G542X mutation as the substrate and using pugIntCFTR as the guide RNA expressing delivery vector, see FIG. 19. Here, the following primers were used: NBD1 PSU-202Fwd: 5′-CTGGAGCCTTCAGAGG-3′ (SEQ ID NO:35) and NBD1PSU+40(491-509)Rev: 5′-GCTCTTGCTAAAGAAATTC-3′ (SEQ ID NO:36).

### Detection of Full-Length Protein by Western-Blot:

The same transfected HEK293T cells that were used for RNA extraction and subsequent RT-PCR (given above) to determine NMD suppression, were used to generate whole cell lysates. These were prepared in 500 μL NET2 buffer (50 mM Tris-HCl, pH 7.5, 150 mM NaCl, 0.05% Nonidet™ P40) supplemented with 0.02% SDS. Cell lysates were applied to sonication (at level 2 for 10 sec) followed by centrifugation (17,000×g for 20 min at 4° C.) to remove cell debris. The supernatant was used as total protein and subjected to protein analysis. Ectopically expressed FLAG-tagged protein was immunoprecipitated from the total protein using Anti-DYKDDDDK Magnetic Agarose (Thermo Scientific). Pulled-down and immunoprecipitated proteins were separated on a 15% SDS-PAGE, immunoblotted, and antibodies were detected by SuperSignal West Femto Maximum Sensitivity Substrate (Thermo Scientific). The FLAG-tagged protein was detected with monoclonal ANTI-FLAG M2, Clone M2 (F1804, SIGMA) as primary antibody and Goat Anti-Mouse IgG (H&L) [HRP], pAb (A10093, Genscript) as secondary antibody. The tubulin housekeeping gene was detected for normalization purposes (primary antibody: Tubulin-beta, Rabbit Polyclonal Antibody #RB-9249-PG (Thermo Scientific); secondary antibody: Anti-rabbit IgG, HRP-linked Antibody #7074S (Cell Signaling)).

Results are shown in FIG. 16. The position of the full length protein FLAG-GL39-IDUA swap is given by an arrow. Lane 1 represents HEK293T cells that were only transfected with the substrate FLAG-GL39-IDUA swap plasmid. Lane 2 represents HEK293T cells transfected with the substrate FLAG-GL39-IDUA swap plasmid+the pugIntronOpt-IDUA plasmid as the guide RNA providing vector. Clearly, there is a significant increase in FLAG-GL39-IDUA abundance, which indicates that pseudouridylation has taken place in the UAG stop codon present in the RNA transcribed from the substrate plasmid and read-through during translation has taken place. This shows that the inventors of the present invention were able to pseudoridylate a target sequence in cells, in vivo, using endogenous (naturally present) RNA editing (pseudouridylation) enzymes, and after providing such cells with a guide RNA that was derived from its natural environment: an intronic sequence.

### Detection of Pseudouridylation

To confirm that the NMD suppression (as detected through RT-PCR, see above and FIG. 15) and the appearance of the full length FLAG-tagged protein (as detected by western blot, see above and FIG. 16) were the result of real pseudouridylation obtained by using a target plasmid containing a premature termination codon and a intronic-carried guide RNA for pseudouridylation, a CMC-modified primer extension method was used that was earlier described by Adachi et al. (2019, Methods Mol Biol 1870:219-235). This multi-step method is based on pseudouridine-specific acylation using a reagent that preferentially modifies the pseudouridine bases, CMC (or CMCT: N-Cyclohexyl-N′-(2-morpholinoethyl) carbodiimide methyl-p-toluenesulfonate). Although bases such as uridine, inosine and guanosine residues are initially also derivatized, it is possible to detect pseudouridines since only these will remain acetylated, while the remaining derivatized bases are hydrolyzed back to their natural chemical form after a mild alkaline treatment step. The way to map the position of the pseudouridines is through a primer extension reaction, where the bulkiness of the CMC at the pseudouridine residue can block the reverse transcription, generating a stop for the primer extension one nucleotide before the CMC-pseudouridine sites. Because natural RT-stops also occur as artifacts generated by strong secondary structures, a negative control (without CMC derivatization) is run in parallel to identify these. This reagent was used to map the position of all known pseudouridine residues in the transcriptome of human cells (Carlile et al, 2014. Nature 515(7525):143-146). For this, HEK293T cells were transfected with substrate FLAG-GL39-IDUA swap plasmid together with either puglntron-IDUA plasmid or with pug-CFTR which served as a negative control, as the CFTR specific guide RNA should not be able to pseudouridylate the IDUA target U in the stop codon. Total RNA was extracted as described above. 20 μg total RNA was used for CMC treatment followed by primer extension with a globin specific primer: hGI193-209AS: 5′-CCGAGCACTTTCTTGCC-3′ (SEQ ID NO:25). 10 μg total RNA was used for primer extension of U6 snRNA control with a U6 specific primer: hU6-86-105AS: 5′-AATATGGAACGCTTCACGAA-3′ (SEQ ID NO:26). The results are given in FIG. 17. The arrow indicates the position of the product where primer extension had stopped because of the presence of a ψ-CMC residue. This only occurred in the cells that were treated with CMC and transfected with the GL39-IDUA swap substrate plasmid and the puglntron-IDUA guide RNA expressing plasmid (zoomed in panel). No bands could be detected in the samples from cells transfected with GL-IDUA swap with the negative control guide RNA expressing pug-CFTR plasmid. The position of the LP residue in relation to the primer used for primer extension is exactly known (92 bases). These results clearly demonstrate that pseudouridylation has taken place in HEK293T cells that were transfected with a target plasmid carrying a premature stop codon in an IDUA context and that were co-transfected with a plasmid carrying—in an intron—a snoRNA that is, once spliced out from the intron sequence, able to target the substrate sequence and to specifically pseudouridylate the U in the UAG stop codon.

### Example 5: Increase in mRNA Levels Upon Treatment with Intron-Imbedded Guide RNAs and psEONs

Further to what has been shown in Example 4, it was then tested whether a psEON, as outlined in detail herein, could also yield pseudouridylation using the substrate GL-IDUA swap plasmids after transfection in cells. For this, HEK293T cells were transfected at 90-100% confluency, using PEI in a 6-well dish, with 500 ng GL-IDUA swap substrate plasmid and 2.5 μg the puglntron-IDUA guide RNA expressing plasmid or transfected with 100 pmol Cy3-IDUA-A psEON oligonucleotide. Four days after transfection cells were washed and incubated at for 24 h. Total RNA was isolated as described and RT-PCR was performed as outlined above, except that 21 cycles were performed for all samples. RT-PCR products were separated by gel electrophoresis. Results are shown in FIG. 18. These indicate that when no DNA was transfected (meaning no plasmid or psEON, on top of the transfected substrate plasmid) that no GL39 RT-PCR product was detectable, although the 5S control was abundant. However, after co-transfection of the pugIntron-IDUA guide RNA-expressing plasmid and also after co-transfection with the Cy3-iDUA-A psEON, the product was detectable, indicating that read-through of the mRNA occurred, and that NMD was inhibited. This shows that the inventors of the present invention were able to obtain pseudouridylation not only by using intronically-embedded guide RNAs, but also with the short psEONs of the present invention.

### Example 6: Design of a Split Pseudouridylating Guide Formed by a Duplex of Oligonucleotides

Previously, it has been shown that it is possible to specifically convert a uridine (U) to a pseudouridine (ψ) in a target RNA by using a single RNA hairpin derived from a H/ACA snoRNA as a guide. The inventors of the present invention questioned whether it would be possible to induce pseudouridylation using a duplex mimicking such a hairpin, formed of shorter oligonucleotides. For this, a previously used pseudouridylation guide RNA design was used as a starting point: This guide was based on the 3′ hairpin of the ACA19 snoRNA, in which the sequence of the pseudouridylation guide pocket was changed so that it could bind the mutated G542X target site within the CFTR mRNA. Designs were then made in which this hairpin was reconstituted from two separate RNA oligonucleotides of varying lengths, thus resulting in a gap in the backbone of the guide in various positions (see FIG. 20). The shortened oligonucleotides were produced by chemical synthesis.

For testing in cell lysates, a short substrate RNA for these guides was produced by two-piece ligation. For the latter, first, a synthetic RNA oligonucleotide, terminating at its 5′ end in the uridine to be pseudouridylated, was radioactively labelled at the 5′ hydroxyl group with a [γ-³²P]phosphate using T4 polynucleotide kinase. The radioactively labelled RNA oligonucleotide was then ligated at its 5′ end to the 3′ hydroxyl group of another RNA oligonucleotide to form the substrate RNA. This was done by annealing the RNA oligonucleotides with a 30-nt bridging DNA oligonucleotide, such that the 5′ half (15 nt) of the bridging DNA oligo base-paired with the 3′ RNA fragment and the 3′ half (15-nt) of the bridging DNA oligo base-paired with the 5′ RNA fragment, and then providing them with T4 DNA ligase to covalently link the RNA oligonucleotides. The ligated, radioactively labelled RNA substrate then has the sequence of 5′-GACAAUAUAGUUCUUUGAGAAGGUGGAAUC-3′ (SEQ ID NO: 37), in which the target uridine (containing the radioactive phosphate on its 5′ side) is underlined. This substrate RNA was then purified from the other nucleic acids by separating it, cutting it from a denaturing polyacrylamide gel, eluting it from the gel and finally concentrating it by ethanol precipitation.

For preparing the cell lysate for testing the pseudouridylation, HeLa cells were grown on standard 10-cm cell culture dishes in DMEM with 10% FBS to a confluency of 80-100%, after which they were collected by scraping, and washed with phosphate buffered saline. Thereafter, 200 μl extraction buffer (25% glycerol, 0.42 M NaCl, 1.5 mM MgCl₂, 0.2 mM EDTA, 20 mM HEPES (pH 7.9), 0.5 mM DTT and 0.5 mM Phenylmethanesulfonyl Fluoride) was added on the cells, which were then vortexed in the presence of sterile glass beads for 30 sec three times, each time followed by a 30 sec incubation on ice. The cell debris was removed by centrifugation, and the supernatant was collected for use in the pseudouridylation assay.

For pseudouridylation assays, substrate RNA and guide RNA oligonucleotides were combined in annealing buffer containing 10 mM Tris-HCl, pH 8.0, 50 mM NaCl, and 1 mM EDTA, heated to 95° C. for 3 mins, and then cooled to room temperature to form the guide RNA-substrate complex. The RNA mixture was then supplemented with incubation buffer containing 200 mM Tris-HCl, pH 8.0, 200 mM ammonium acetate, 10 mM MgCl₂, 4 mM DTT, and 0.2 mM EDTA and HeLa cell extract (final concentration 20%). The mixture was incubated at 42° C. for 30 min. RNA was then isolated from the reactions by phenol-chloroform extraction and ethanol precipitation. The RNA was then incubated with P1 nuclease (~300 ng) in 20 mM sodium acetate buffer, pH 5.2 for 1 h at 37° C. to degrade the RNA and release the individual nucleotides. These were then separated by thin-layer chromatography (with the solvent volume ratios 70:15:15 for isopropyl alcohol:HCl:water respectively), and the radioactive nucleotides were then imaged by autoradiography. Due to the different migration of the labelled uridines and pseudouridines derived, the conversion of the labelled substrate uridine into pseudouridine can be clearly observed. FIG. 21 shows that when the duplex pseudouridylating guides formed from two oligonucleotides are used, pseudouridylation of the substrate RNA can be observed. The efficiency of the conversion varies depending on the location of the gap in the duplex, at least for the substrate and guides used in this experiment.

### Example 7: Design of a Split Pseudouridylating Guide Formed by a Duplex of Chemically Modified Oligonucleotides

Then, it was investigated whether chemical modifications made in the ACA19 guide duplex would be compatible with the target RNA engagement, and the formation of a catalytically active pseudouridylation complex. The positions of the chemical modifications were selected as described herein. The substrate RNA and the experimental assays used were the same as in Example 1, except for the cell extract, which in this case was derived from HEK293 cells, and used at the final concentration of 10%. FIG. 22 shows two full-length ACA19 pseudouridylating editing oligonucleotides (psEONs) and the five duplexes formed from shorter psEONs. The psEONs vary in their chemical compositions, and positions of the chemical modifications are indicated as follows: lower case letters (a, c, g and u) indicate 2′-OMe modifications on the ribose moiety of that nucleotide, and a lowercase letter s indicates PS modification of the linkage between the two riboses. The duplex psEONs also vary in their base composition, such that the duplexes have varying levels of internal base-pairing interactions.

FIG. 23 shows the result of pseudouridylation using the five duplex psEONs in parallel with the corresponding full-length psEONs as a positive control and no guide RNA as a negative control. U and ψ indicate the migration of uridine and pseudouridine in the thin layer chromatography, respectively. Clearly, the duplex psEONs were able to support conversion of uridine to pseudouridine, similar to the full-length psEONs. As synthesis of two short oligonucleotides is much easier to optimize than that of a single long oligonucleotide, this can result in advantage for the development and production of therapeutic compounds based on pseudouridylating guides.

1. A composition comprising two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme.

2. The composition according to claim 1, wherein the pseudouridylation enzyme is part of a ribonucleoprotein (RNP) complex capable of acting on an H/ACA-snoRNA.

3. The composition according to claim 2, wherein the two or more oligonucleotides form a single guide region corresponding to one of the two hairpin structures of the wild type H/ACA snoRNA.

4. The composition according to claim 3, wherein at least one of the two or more partially duplexed oligonucleotides consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

5. The composition according to claim 1, wherein the partially duplexed nucleic acid molecule comprises one or more nucleosides or inter-nucleosidic linkages that are non-naturally modified.

6. The composition according to claim 5, wherein the non-natural modification comprises a modified sugar moiety.

7. The composition according to claim 6, wherein the modified sugar moiety is selected from the group consisting of 2′-deoxy, 2′-O-alkyl, 2′-halogen, and 2′-O-methoxyethyl (2′-MOE).

8. The composition according to claim 5, wherein one or more non-natural inter-nucleosidic linkages is selected from the group consisting of phosphodiester linkages, phosphorothioate (PS) linkages, phosphorodithioate linkages, phosphotriester linkages, and methylphosphonate (MP) linkages, or any combination thereof.

9-12. (canceled)

13. The composition according to claim 1, wherein the target RNA comprises a mutation that is associated with a genetic disorder.

14. The composition according to claim 13, wherein the mutation results in a Premature Termination Codon (PTC), wherein the PTC is the cause of the genetic disorder, and wherein the target uridine is in the PTC.

15. A method of treating, preventing, delaying, or ameliorating a disease or disorder in a subject comprising administering the composition of claim 1 to the subject, wherein the disease or disorder is selected from Cystic fibrosis, Hurler Syndrome, alpha-1-antitrypsin (A1AT) deficiency, Parkinson's disease, Alzheimer's disease, albinism, Amyotrophic lateral sclerosis, Asthma, 3-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase deficiency, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, or cancer.

16. A method for converting a uridine in a target RNA molecule into a pseudouridine, comprising the steps of contacting the target RNA comprising the target uridine with the composition of claim 1 in the presence of a pseudouridylation enzyme or RNP complex and allowing the uridine to be converted thereby.

17. The method of claim 16, comprising administering the composition to a mammalian cell comprising the target RNA comprising the target uridine.

18-19. (canceled)

20. A composition comprising one or more vectors, wherein the one or more vectors encode two or more oligonucleotides, wherein the two or more oligonucleotides associate to form a partially duplexed nucleic acid molecule for pseudouridylation of a target uridine in a target RNA in a mammalian cell, wherein the partially duplexed nucleic acid molecule comprises a single stranded guide region capable of forming a complex with the target RNA comprising the target uridine, wherein the partially duplexed nucleic acid molecule when complexed to the target RNA is capable of engaging a mammalian pseudouridylation enzyme, wherein the guide region assists in positioning the target uridine to be converted to a pseudouridine by the mammalian pseudouridylation enzyme, and wherein each of the one or more vectors comprise an intron sequence that is located between an upstream exon A sequence and a downstream exon B sequence, wherein the exon A sequence and the exon B sequence are of a gene that is not the natural gene for the intron sequence, and wherein the intron sequence of each of the one or more vectors encode at least one of the two or more oligonucleotides.

21. The composition of claim 20, wherein the composition comprises:
a. a single vector, and wherein the intron of the single vector encodes the two or more oligonucleotides, or
b. two or more vectors, and wherein each vector comprises an intron encoding one of the two or more oligonucleotides.

22. (canceled)

23. The composition according to claim 20, wherein the exon A sequence is exon 1 of the human β-globin gene, and the exon B sequence is exon 2 of the human β-globin gene.

24. The composition according to claim 20, wherein the vector is a plasmid or a viral vector.

25. A method of treating, preventing, delaying, or ameliorating a disease or disorder in a subject comprising administering the composition of claim 20 to the subject wherein the disease or disorder is selected from Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase deficiency, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome, X-linked immunodeficiency, Sturge-Weber Syndrome, or cancer.

26. A pharmaceutical composition comprising the composition of claim 20, and one or more of a pharmaceutically acceptable carrier, stabilizer or solvent.

27. A method for converting a uridine in a target RNA molecule into a pseudouridine in a cell, comprising the steps of:
administering to the cell the composition of claim 20;
allowing the transcription of the one or more exon A/intron/exon B sequence;
allowing splicing and the formation of the two or more oligonucleotides positioned in the intron;
allowing the two or more oligonucleotides to associate to form a partially double stranded nucleic acid molecule; and
allowing the partially double stranded nucleic acid molecule to form a complex with the target RNA molecule,
wherein the complex is capable of engaging a mammalian pseudouridylation enzyme to form a functional RNP complex, wherein the snoRNA correctly positions the target uridine for it to be converted by the RNP complex, and wherein the target uridine is converted by the RNP complex to a pseudouridine.

28. (canceled)
